(19) United States
(12) Patent Application Publication
(10) Pub. No.: US 2011/0052671 A1
(43) Pub. Date: Mar. 3, 2011

(54) NEAR INFRA-RED PULSED LASER TRIGGERED DRUG RELEASE FROM HOLLOW NANOSHELL DISRUPTED VESICLES AND VESOSOMES

(75) Inventors: Joseph R. Zasadzinski, Santa Barbara, CA (US); Guohui Wu, Seattle, WA (US); Brian Prevo, Arlington, MA (US)

(73) Assignee: THE REGENTS OF THE UNIVERSITY OF CALIFORNIA, Oakland, CA (US)

(21) Appl. No.: 12/863,010

(22) PCT Filed: Jan. 30, 2009
(86) PCT No.: PCT/US09/32534

§ 371 Date: Jul. 15, 2010

Related U.S. Application Data
(60) Provisional application No. 61/024,690, filed on Jan. 30, 2008.

(51) Int. Cl. A61K 9/127; A61K 9/14; A61K 9/133; A61K 39/44; B82Y 5/00; B82Y 40/00

(52) U.S. Cl. 424/450; 424/489; 424/178.1; 977/700; 977/906; 977/773; 977/810; 977/895; 977/762

(57) ABSTRACT

The disclosure provides drug delivery methods and compositions. More particularly, the application provides liposomal delivery compositions comprising a nanostructure.

### CROSS-REFERENCE TO RELATED APPLICATIONS

This application claims priority under 35 U.S.C. §119 to U.S. Provisional Application Ser. No. 61/024,690, filed Jan. 30, 2008, the disclosure which is incorporated herein by reference.

### STATEMENTS REGARDING FEDERALLY SPONSORED RESEARCH

The invention was funded in part by Grant No. 1U01 HL080718-01 from the National Institutes of Health (NIH) and by U.S. Army Grant No. DAAD19-03-D-0004. The government has certain rights in the invention.

### TECHNICAL FIELD

The disclosure provides methods and compositions for the generation of nanoparticles. The disclosure also relates to drug delivery methods and compositions. More particularly, the application relates to liposomal delivery comprising a nanostructure.

### BACKGROUND

Effective drug delivery is important to reduce dosing and side effects.

### SUMMARY

The disclosure provides a method of generating monodisperse hollow metal nanostructures comprising: contacting a template metallic nanostructure stabilized by biocompatible anions in an aqueous environment with a noble metal salt precursor having greater standard reduction potential than the template metallic nanostructure.

The disclosure also provides a method of generating monodisperse hollow metal nanostructures comprising: providing a template metal nanostructure; modifying the size of the template metal nanostructure; stabilizing the template metallic nanostructure with a biocompatible anion to provide a stabilized template metallic nanostructure; adding a noble metal salt precursor to the stabilized template metallic nanostructure, wherein the noble metal salt precursor comprises a greater standard reduction potential than the template metallic nanostructure.

The disclosure further provides nanostructure generated by the methods of the disclosure, dispersions of such nanostructure and preparations thereof.

The disclosure provides a composition comprising: a liposome; a therapeutic or diagnostic agent encapsulated within the liposome; a nanostructure; wherein the nanostructure is encapsulated within the liposome or tethered to the liposome or mixed with the liposome solution, wherein the nanostructure can absorb electromagnetic radiation and generate vibrational or thermal energy from the electromagnetic radiation. In one embodiment, the liposome is selected from the group consisting of an MLV, an MVL, a ULV and a vesosome. In another embodiment, the nanostructure is a metallic nanostructure. In yet another embodiment, the metallic nanostructure is a nanoshell, such as a hollow gold nanoshell. However, the nanostructure can comprise any metallic or metallic alloy material. Furthermore, the nanostructure can have any geometry (e.g., a shell, a crescent, a particle, or a rod). In yet a further embodiment, the liposome can further comprise a targeting moiety linked to the liposome. The targeting moiety can be, for example, an antibody, an antibody fragment, a receptor or a receptor ligand. In yet a further embodiment, the nanostructure can comprise a targeting moiety linked to the nanostructure. In one embodiment, the therapeutic agent is a chemotherapeutic agent. In another embodiment, the composition further comprises a pharmaceutically acceptable carrier. In another aspect, the agent can be a diagnostic agent. In another aspect, the agent can be an image enhancing agent for X-ray or MRI or ultrasound imaging.

The disclosure further provides a formulation comprising: a liposome; a therapeutic or diagnostic agent encapsulated within the liposome; a nanostructure; wherein the nanostructure can absorb electromagnetic radiation and generated vibration or thermal energy from the electromagnetic radiation.

The disclosure also provides a method for delivery of an agent to a subject or tissue, comprising: contacting the subject or tissue with a composition, formulation or pharmaceutically acceptable composition as described above; and contacting a desired location on the subject or tissue with an electromagnetic radiation comprising a wavelength that induces vibrational or thermal energy of the nanostructure for a sufficient time to cause a liposome in the composition to be disrupted. In one embodiment, the liposome is selected from the group consisting of an MLV, a MVL, a ULV and a vesosome. In another embodiment, the nanostructure is a metallic nanostructure. In yet another embodiment, the metallic nanostructure is a nanoshell, such as a gold nanoshell. However, the nanostructure can comprise a metallic nanostructure that can have absorbance, in particular a strong absorbance, in the wavelength range from 500-1000 nm, typically in the range from 650 nm-900 nm. Furthermore, the nanostructure can have any geometry (e.g., a shell, a crescent, a particle, or a rod). In yet a further embodiment, the liposome can further comprise a targeting moiety linked to the liposome. The targeting moiety can be, for example, an antibody, an antibody fragment, a receptor or a receptor ligand. In one embodiment, the therapeutic agent is a chemotherapeutic agent. In another embodiment, the composition further comprises a pharmaceutically acceptable carrier. In one embodiment, the electromagnetic radiation comprises near-infrared radiation. In another aspect, targeting to specific areas within the body is done by focused near infrared laser light.

This disclosure also provides for a method of releasing drugs at specific areas within the body via focused laser light. Only nanostructures directly irradiated will trigger release from the liposomes, providing a chemically free and externally actuated method of localizing drug release. Such release can reduce the side effects and improve the efficacy of a range of drugs without the need of developing ligands or antibodies specific to a particular tissue or area within the body.

### DESCRIPTION OF DRAWINGS

FIG. 1A-D shows TEM images of compositions of the disclosure and spectra data.(a) Cryo-TEM images showing dark, spheroidal hollow gold nanoshells (HGNs, arrows) of ˜35 nm diameter encapsulated inside liposomes formed via the interdigitated phase. The HGNs have a bright spot in their center in the images, indicative of a hollow core. Unilamellar liposomes appear as a dark grey ring enclosing a light grey interior in cryo-TEM images. The liposome membranes are almost perfectly circular, indicating that the membrane is under tension due to the higher CF concentration inside the liposome, which generates a higher osmotic pressure inside the liposome than outside. (b) Hollow gold nanoshells tethered to DPPC liposomes via a thiol-PEG-lipid linkage; untethered HGNs were removed by centrifugation. (c) HGN added to already formed liposomes, but not encapsulated or tethered to the liposomes. All of the liposomes containing CF appeared nearly circular in the TEM images consistent with an osmotic pressure higher in the liposome inducing a membrane tension. (d) UV/vis spectra of HGNs with the surface plasmon resonance maximum absorption at 820 nm.

FIG. 2A-B shows fluorescence and electromagnetic irradiation of compositions of the disclosure. (a) In situ fluorescence intensity measured during NIR laser irradiation of DPPC liposomes (0.2 mg/ml) encapsulating HGNs (12 mM gold concentration in solution) and CF dye (16 mM inside liposome) with 130 fs pulses of 800 nm light at a 1 kHz pulse rate (the Gaussian beam width was 2.3 mm) as a function of laser power density. Time zero is the beginning of laser irradiation. Below a threshold power density (˜1.5 W/cm², see FIG. 2b), the fluorescence intensity was constant. Above the threshold, there was a near instantaneous increase in the fluorescence intensity, followed by a more gradual increase. The solid lines are single exponential fits,

$$F = {F}_{o} + A {e}^{- \frac{x}{τ}} ,$$

to the data; τ=5, 52 and 112 at laser power densities of 14.9, 13.0 and 7.1 W/cm², respectively. (b) Effect of laser power (red curve) on the fractional release of CF from irradiated DPPC liposomes (0.2 mg/ml) encapsulating HGN (12 mM gold concentration in solution) and 16 mM CF dye inside the liposomes. The solid curve is a sigmoidal fit to the fractional release:

$$y = \frac{{y}_{max} - {y}_{min}}{1 + {e}^{( E - {E}_{o} ) / Δ E}} + {y}_{min}$$

with y_{max, min} the upper and lower values of the release, Eₒ, the threshold of the NIR laser power and ΔE the width of the sigmoid. The green curve is the effect of laser power on the fractional release of CF from DPPC liposomes (0.2 mg/ml) with 12 mM gold concentration in solution, but not encapsulated within the liposomes. The total release is smaller, but the threshold power for release is about the same. This suggests that a minimum energy must be absorbed by the HGN to induce cavitation; however, the effects of the cavitation bubbles depend on the proximity of the HGN to the liposome membrane.

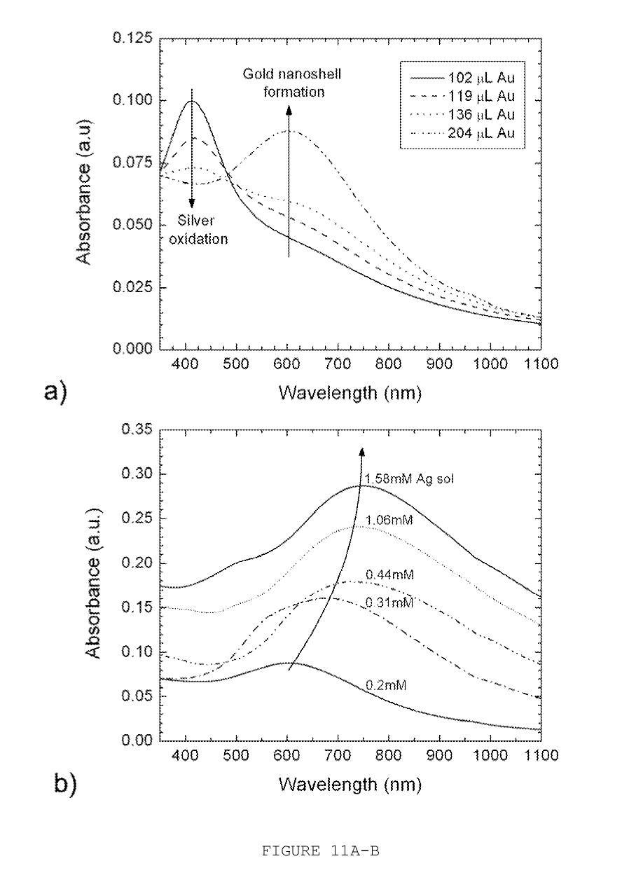

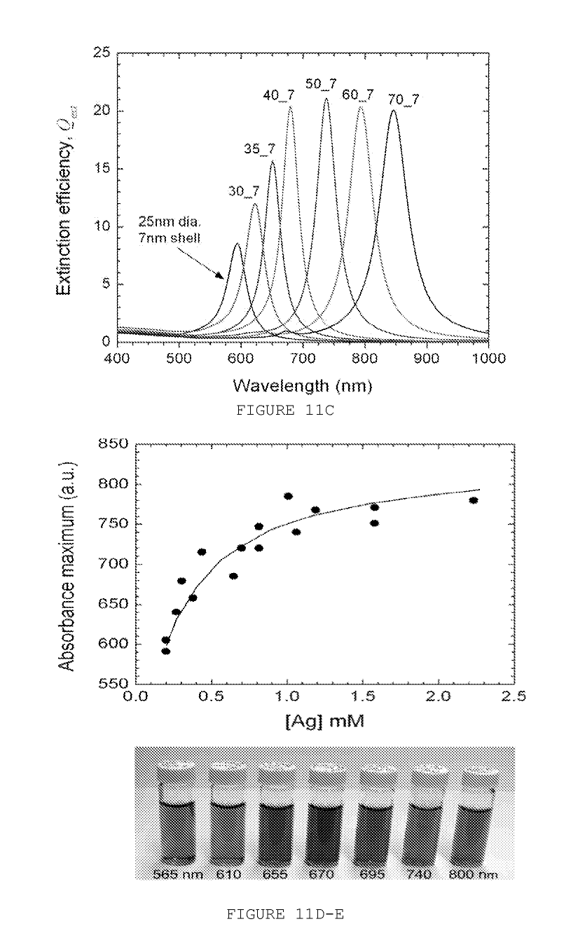

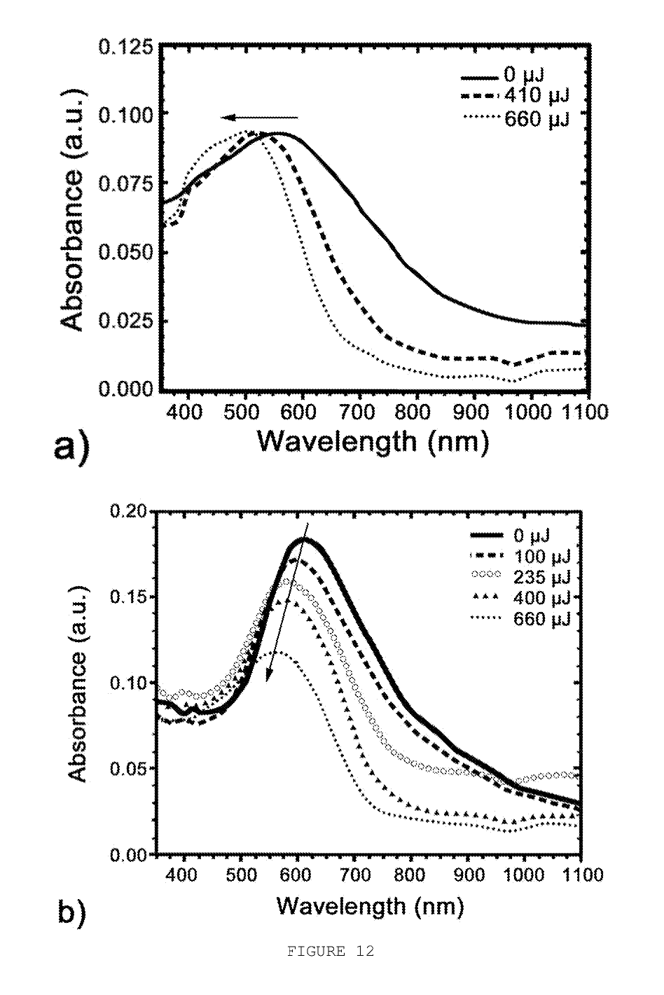

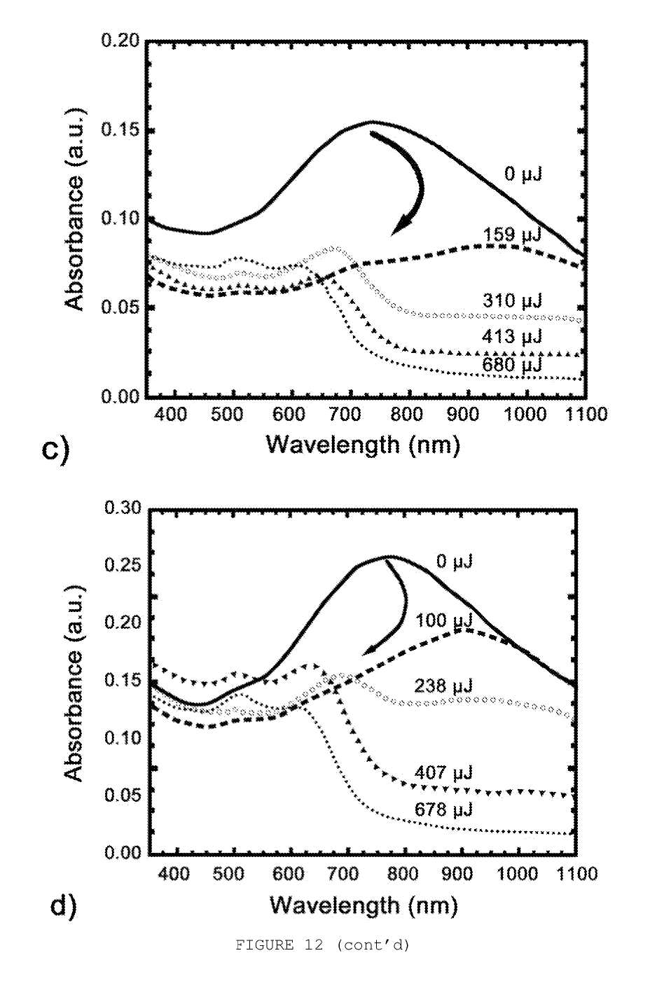

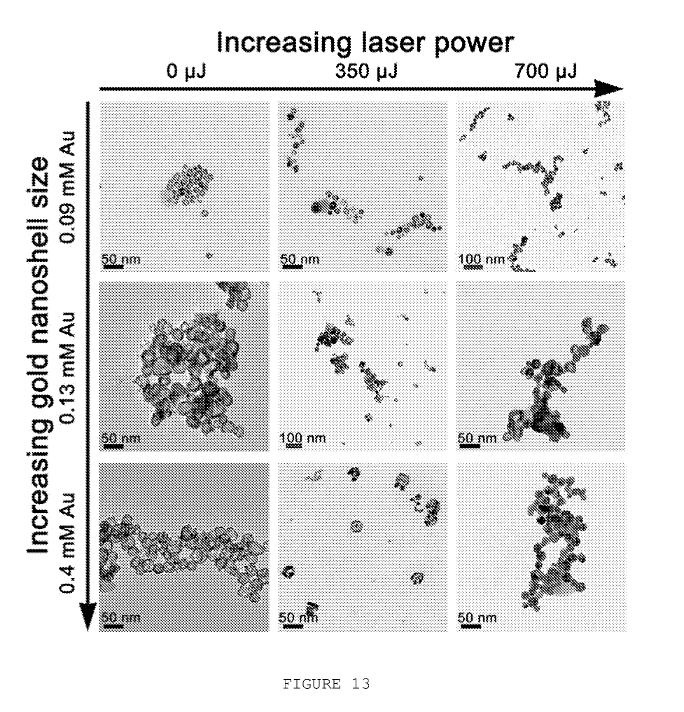

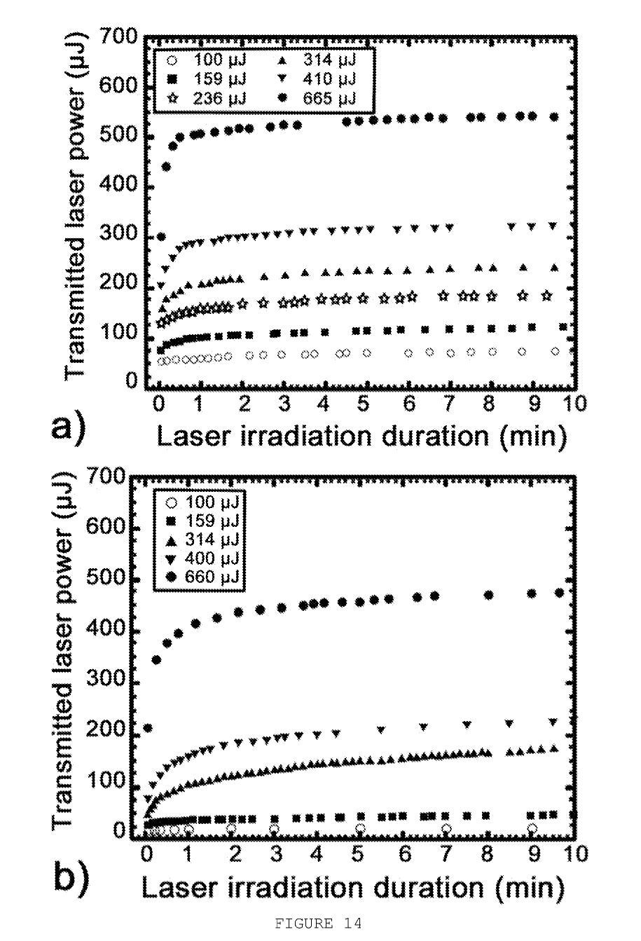

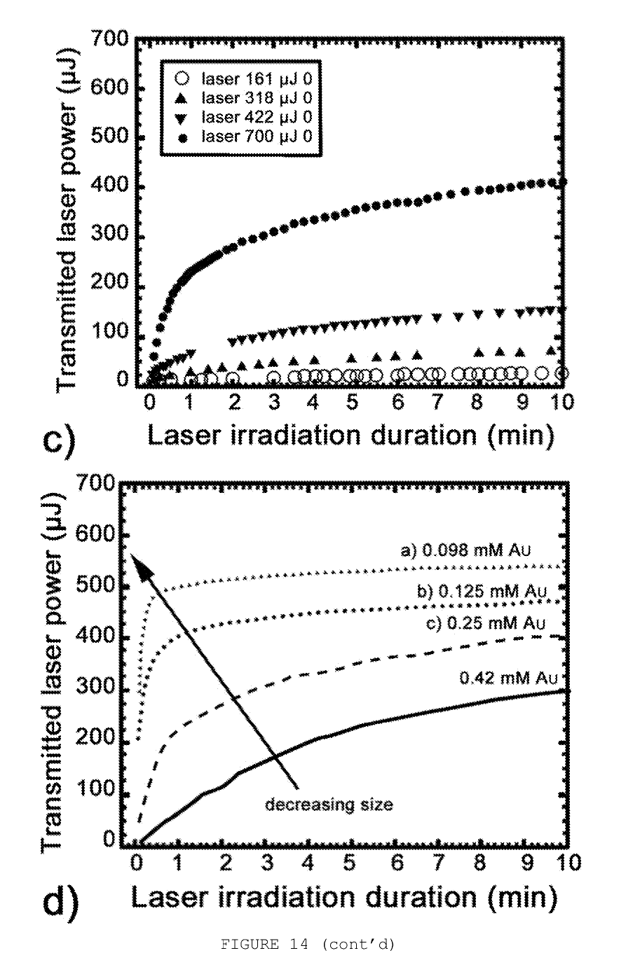

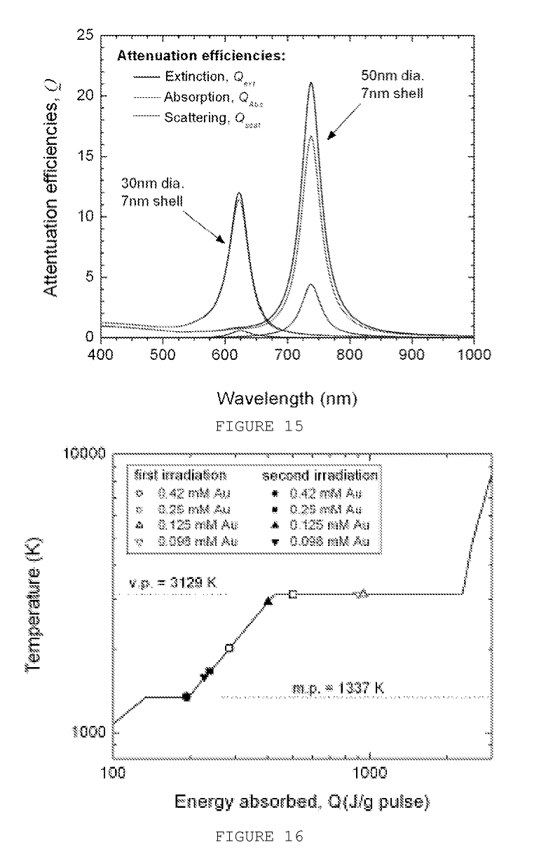

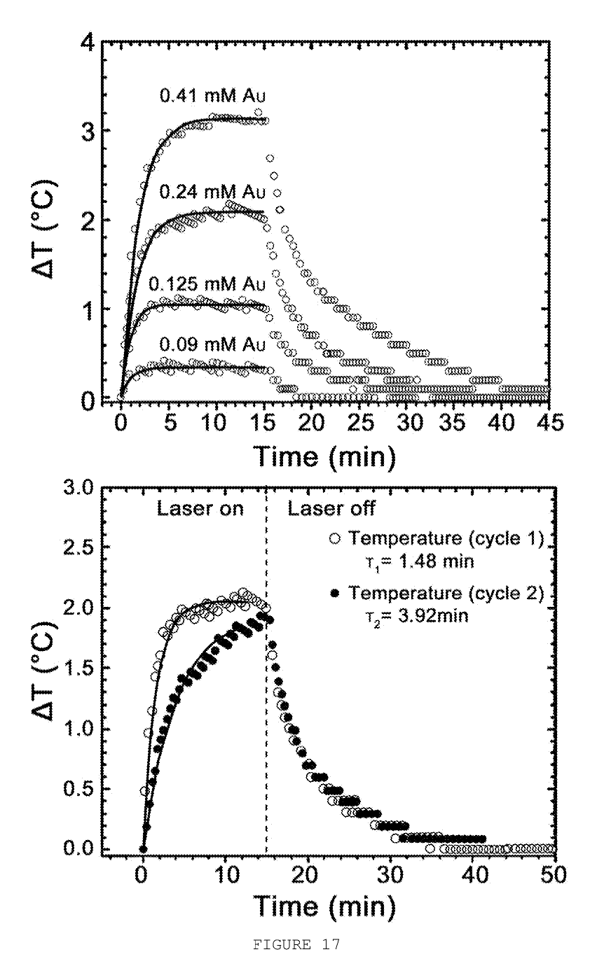

FIG. 3A-B shows photoaccoustic signals and laser density of compositions of the disclosure. (a) Typical photoaccoustic signal of pressure fluctuations similar to cavitation recorded by a hydrophone from a 0.142 mM HGN solution triggered by a 130 fs laser pulse of power density 16.1 W/cm² at time zero. The inset is an enlarged view of the first 250 μsec. (b) Effect of laser power density on the amplitude of cavitation signals recorded by the hydrophone. A threshold value of laser power density 2.3 W/cm² is observed to induce the cavitation signals above background, which is similar to the threshold laser power density to trigger the content release from liposomes in FIG. 2b. The amplitude of the pressure fluctuations increases with increasing laser power density; as the HGNs absorb more light, their maximum temperature increases, which translates into larger pressure fluctuations as this energy dissipates into the solution.

FIG. 4A-B shows fractional release from compositions of the disclosure. (a) Increasing the concentrations of un-encapsulated HGNs increases the fractional release of CF from DPPC liposomes (0.2 mg/ml). The liposomes were exposed to the NIR laser at 16.3 W/cm² for 9 minutes. The solid line is a linear fit to the data. (b) The proximity of the HGN to the lipid membrane strongly influences the fractional release of CF. HGNs directly tethered to the bilayer induce 96% CF release; encapsulated HGNs induce 74% release and HGNs in solution, but not encapsulated, induce 30% release on irradiation with 130 femtosecond laser pulses. However, continuous radiation at the same power density causes no CF release from the liposomes.

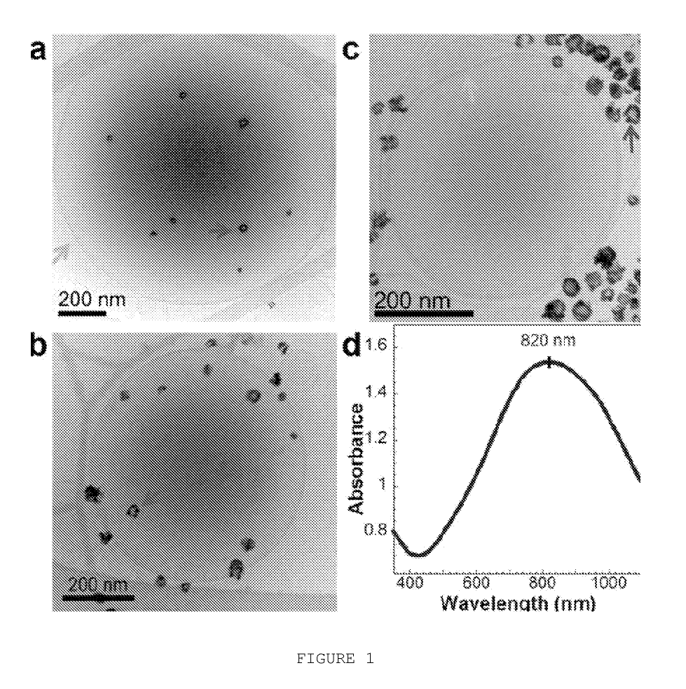

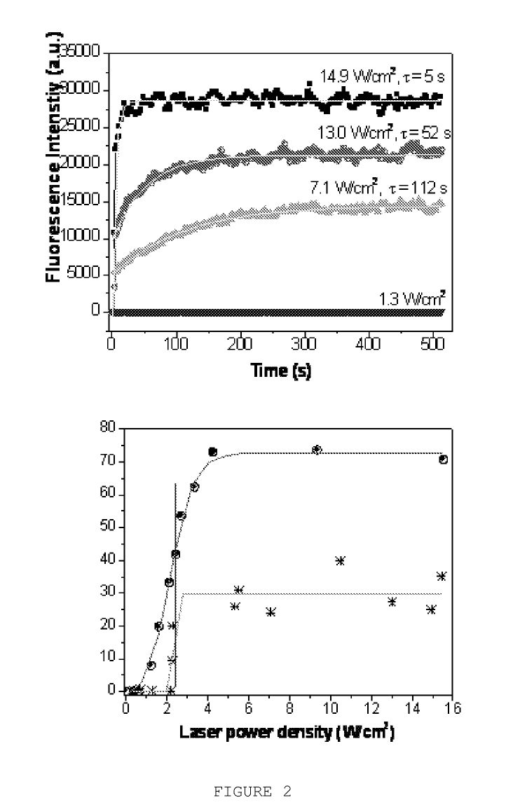

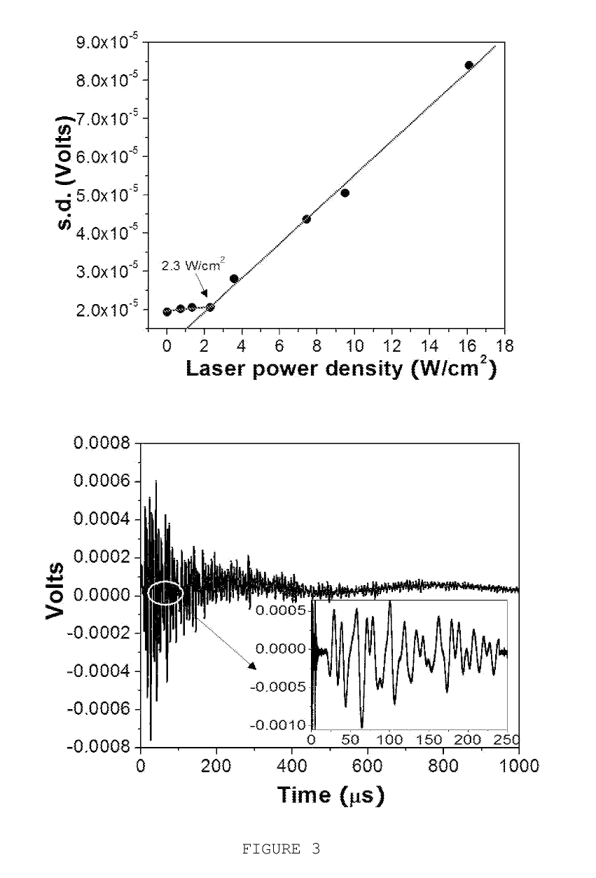

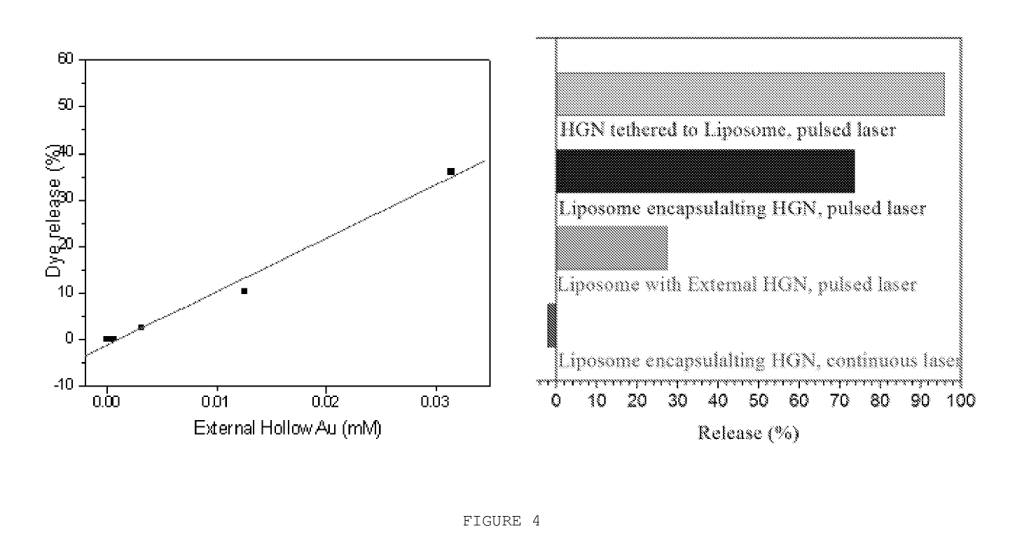

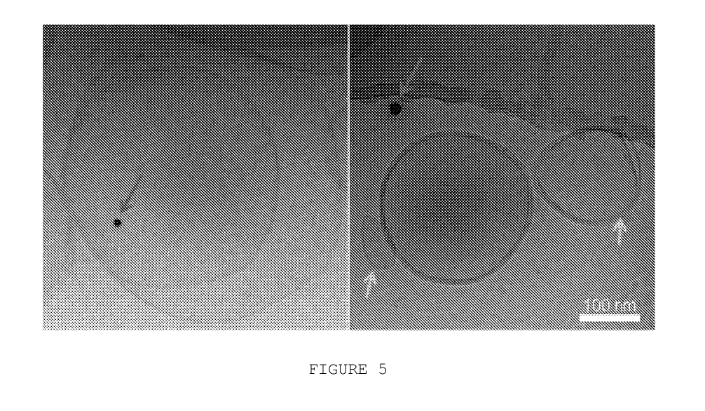

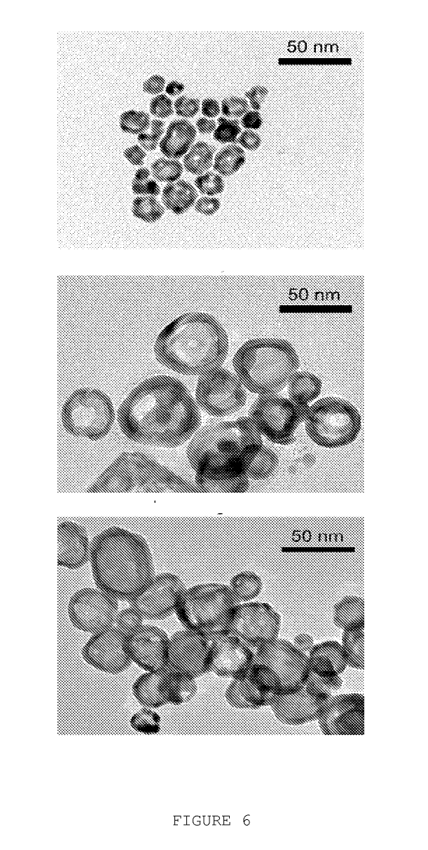

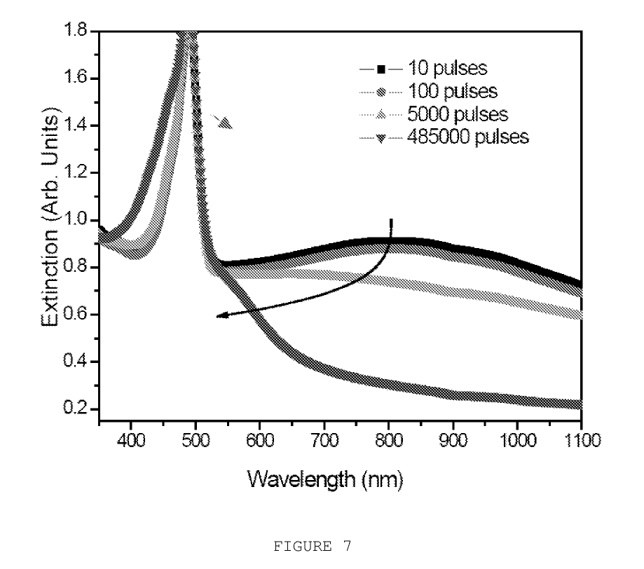

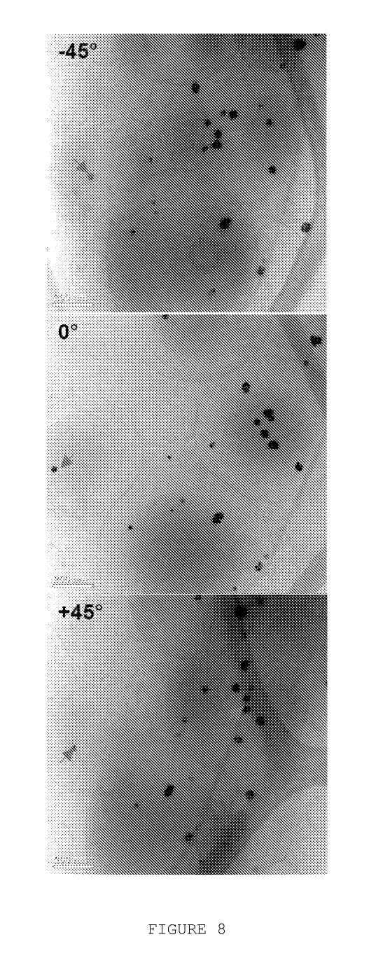

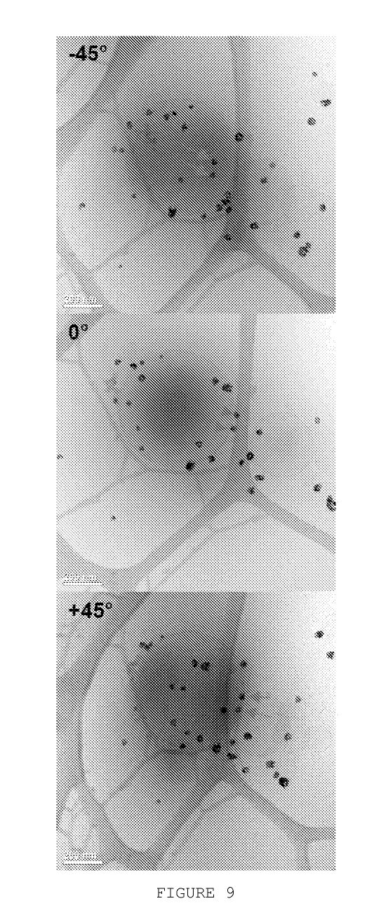

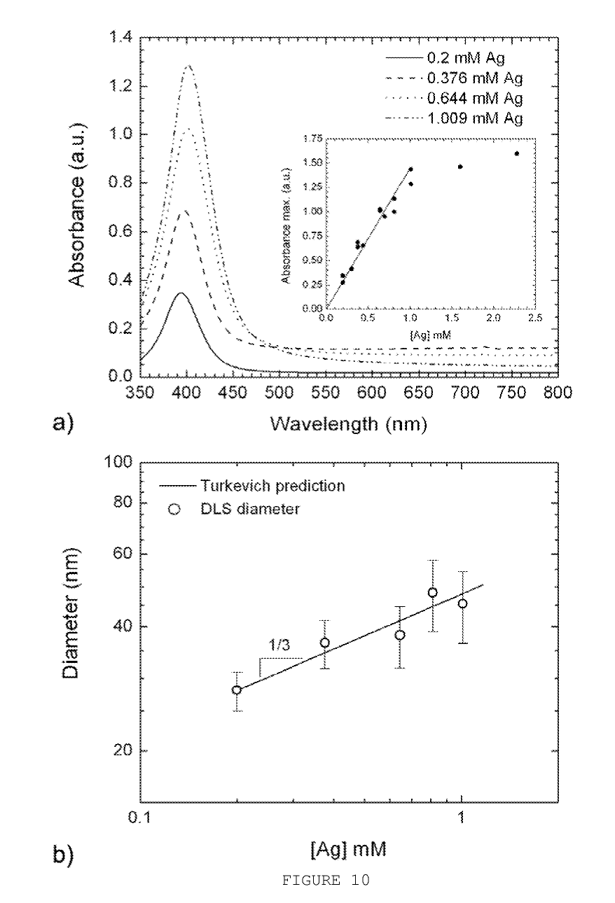

FIG. 5 shows cryo-TEM images of the change in the HGN morphology from a hollow core (See FIG. 1) to a solid core nanoparticle (red arrows) after 16.1 W/cm² NIR laser irradiation, both inside (left) and outside (right) of the liposomes. There are only subtle changes in the liposome morphology in comparison to FIG. 1. The bilayers are no longer completely circular (arrows) consistent with the release of the interior CF and relaxation of the osmotic pressure difference, which removes the membrane tension.

FIG. 6 shows TEM images of gold nanoshells made from the unmodified ˜20 nm Ag sol (0.2 mM Ag) and those made from the growth sol in which the concentration of silver was increased. Top: Unmodified 0.2 mM Ag sol. Center: 0.304 mM Ag. Bottom: 0.391 mM Ag. The gold nanoshell size depends on the size of the sacrificial silver template. The center of each nanoshell is relatively electron-transparent, indicative of a hollow center showing that the silver template nanoparticle was converted to molecular silver.

FIG. 7 shows a UV-Vis spectrophotometry of the suspension of DPPC liposomes (0.2 mg/ml), encapsulating HGNs and CF dye (16 mM inside liposome), to 16 W/cm² laser irradiation at 800 nm. The surface plasmon peak of the HGN at ˜820 nm steadily decreases with increasing irradiation, consistent with the melting and annealing of the nanoshells into solid nanoparticles (See FIGS. 5 and 13).

FIG. 8 shows cryo-TEM images of the same set of liposomes with encapsulated HGN taken at different goniometer tilt angles: −45°; 0°; +45°. The sample contains 22 mg/ml DPPC liposomes encapsulating 12 mM HGN and 16 mM CF inside. The red arrows point out the same HGNs during the 90° range tilt to confirm that HGNs are inside the liposomes.

FIG. 9 shows cryo-TEM images of the same set of liposomes with HGN tethered to the outer surface of the bilayer taken at different goniometer tilt angles: −45°; 0°; +45°. The sample contains 18 mM HGN, 18 mg/ml phospholipids (98 mole % DPPC and 2 mole % DSPE-2000PEG-SH) containing 16 mM CF inside. The red arrows are examples to mark the HGNs followed during the 90° tilt to confirm that HGNs are tethered to the liposome surface.

FIG. 10A-B shows spectra and light scattering of compositions of the disclosure. (a) UV/visible spectra of 10-fold diluted silver sols made from 0.2 mM Ag stock suspensions, to which additional silver nitrate was added. Inset: surface plasmon resonance (SPR) behavior of the silver sols (stock and growth) as a function of the final silver concentration. The dilutions were done so that accurate measurements could be obtained. Beer's law held (for the diluted samples) until ˜1 mM. (b) Dynamic light scattering (DLS) measured diameters of the stock silver sol (0.2 mM Ag) and the growth sols. The growth sol diameter scaled with the ⅓ power of the silver concentration confirming that all of the silver added to existing nanoparticles and did not nucleate new particles.

FIG. 11A-E shows data on compositions of the disclosure. (a) Ultraviolet/visible light spectrophotometry of the conversion of a 0.2 mM Ag sol to a 0.09 mM Au nanoshell suspension by adding increasing amounts of 25 mM HAuCl₄. For clarity only four curves are shown. (b) The resulting final absorbance spectra for gold nanoshells made from different sized silver sols (denoted by their initial silver concentration) after 10-fold dilution. The higher the silver concentration, the larger the silver template, the larger are the resulting HGN's, and the absorbance of the final gold nanoshells is red-shifted to longer wavelengths. (c) Calculated extinction efficiencies for different sizes of gold nanoshells with a water core and water surroundings. The first number is the overall diameter of the nanoshell and the second number is the shell thickness (i.e., 30−7 means a nanoshell of 30 nm diameter with a 7 nm shell). Increasing the nanoshell diameter for a given shell thickness shifts the surface plasmon resonance to longer wavelengths and increases the extinction efficiency. (d) A summary of the dependence that the final gold nanoshell SPR has on the initial sacrificial silver sol (size and concentration). Each silver template started from a stock 0.2 mM silver sol, and was grown from that to larger particles by adding more AgNO₃. The error in each point (sample to sample variation) is on the order of ±15 nm (primarily due to the polydispersity in the initial silver template), but they are omitted for clarity. The curve is a guide the eye. (e) A collection of different aqueous gold nanoshell dispersions prepared from silver sols of increasing concentration and particle size (from left to right). The average SPR absorbance maximum for a given sol is listed beneath each vial. The overall range is from ˜560-800 nm, showing that the absorbance profiles of gold nanoshells made via this process can be tuned across the NIR window.

FIG. 12A-D shows UV/visible light spectra for different gold nanoshells suspensions after 10 minutes of exposure to 800 nm pulsed laser irradiation. (a) 0.09 mM Au gold nanoshells from an initial 0.2 mM Ag sol, (20 nm diameter, surface plasmon resonance, SPR ˜600 nm), (b) 0.124 mM Au gold nanoshells from a 0.304 mM Ag sol (30 nm diameter, SPR ˜650 nm), (c) 0.25 mM Au gold nanoshells from a 0.675 mM Ag sol (40 nm diameter, SPR ˜720 nm), and (d) 0.41 mM Au gold nanoshells from a 1.07 mM Ag sol (50 nm diameter, SPR ˜750 nm). The arrows are in the direction of increasing laser power. A fresh, un-irradiated sample was used in each trial. The absorbance increases (from a-d) with increasing gold/silver concentrations as expected from FIGS. 11 and 6; the higher concentrations lead to larger gold nanoshells with the surface plasmon resonance closer to the 800 nm irradiation wavelength. After 10 minutes of irradiation, all samples show a peak from 500-600 nm consistent with the formation of solid, spherical nanoparticles as seen in FIG. 14.

FIG. 13 shows TEM images of three different concentrations, 0.09 mM, 0.13 mM, and 0.41 mM Au nanoshells before and after 10 minutes of irradiation with 800 nm light pulses of 350 or 700 μJ total energy. After irradiation with the higher energy pulses, all of the nanoshells have sintered and annealed resulting in 10-40 nm diameter solid spheres; the larger nanoshells anneal into larger spherical particles. At 350 μJ, the smaller particles from the 0.09 mM sol have converted to the smaller diameter solid spheres. However, the larger particles from the 0.13 and 0.41 mM sols first break apart upon absorbing the lower photon energy, leading to a disperse collection of asymmetric incomplete shells, oblate spheroids, rods and branched structures. The optical holes seen in FIGS. 12c,d may be the result of these particle shapes that exhibit SPR shifted to longer wavelengths.

FIG. 14A-D shows laser light transmission as a function of irradiation time for varying sizes of gold nanoshells: (a) 20 nm diameter (0.098 mM Au), (b) 30 nm (0.125 mM Au), (c) 40 nm (0.25 mM Au). The initial transmitted intensity is highest for the smallest nanoshells, consistent with the smallest overlap of the SPR peak (FIG. 11) with the 800 nm wavelength of the laser light. The transmitted intensity increases with time as expected from the spectra in FIG. 13 that show the nanoshells eventually are melted and annealed into solid spherical nanoparticles with less absorption at 800 nm (FIG. 12). (d) Highest (700 μJ/pulse) energy laser absorbance kinetics comparison for the nanoshells in (a), (b), (c) and for ˜50 nm diameter gold nanoshells (0.42 mM Au). The larger nanoshells anneal much more slowly than the smaller nanoshells, resulting in lower light transmission after 15 minutes.

FIG. 15 are graphs showing Mie calculations of the relationship between scattering, absorption (middle curves) and total extinction (highest curves) as a function of particle size. The larger particles scatter a larger fraction of the light and hence are less efficient absorbers, although the wavelength of the peak absorption overlaps more strongly with the excitation wavelength (800 nm) of the incident NIR radiation. The relative adsorption efficiency (ε_{A} in Eqn. 2) is the ratio of the absorption to the total extinction and is ˜0.95 for the 30 nm nanoshells and ˜0.8 for the 50 nm diameter nanoshells.

FIG. 16 is a graph showing estimate of maximum nanoshell temperature upon initial irradiation (open symbols) and after 10 minutes irradiation (closed symbols) with maximum laser power (˜690 μJ/pulse) using Eqns. 2, 3 and total extinction determined from FIG. 15 and efficiencies from FIG. 16. The different concentrations correspond to different nanoshell diameters: 0.098 mM Au: 20 nm, 0.125 mM Au: 30 nm, 0.25 mM Au: 40 nm, and 0.42 mM Au: 50 nm. While the extinction peak for the smaller nanoshells (lower Au concentrations) does not overlap with the incident laser wavelength as well as that for the larger nanoshells (FIG. 15), the proportionately greater increase in mass of the larger nanoshells leads to a smaller increase in the maximum temperature.

FIG. 17A-B shows calorimetric data of compositions of the disclosure. a) Experimental calorimetric data showing the degree of heating due to different 3 ml total volume gold nanoshell suspensions after being irradiated for 15 minutes with 680 μJ/pulse energy at 1 kHz (0.68 W power). These decreasing concentration of gold correspond to (a) ˜20 nm, (b) ˜30 nm, (c) ˜40 nm, and (d) 50 nm nanoshells. The largest particles adsorb the largest total energy as shown by the net increase in water temperature. The steady state increase in temperature for the nanoshells scales roughly with the gold mass ratio for the larger nanoparticles; the 0.24 mM nanoshell suspension temperature increase is roughly twice that (2° C. vs 1° C.) of the 0.125 mM nanoshell suspension. (b) Calorimetry of resonance and off resonance heating via laser absorption for the a nanoshell suspension which had an SPR of ˜750 nm initially (open circles), and after melting was subsequently cooled and then irradiated again (closed circles). The initial rise in temperature is much faster for the first irradiation, but levels off to roughly the same steady state temperature as the second irradiation. This is because the nanoshells are being transformed during the first irradiation to smaller, solid gold nanoparticles (FIG. 13, 14) that adsorb less light energy. By the time steady state is reached, most of the nanoshells have been converted to nanoparticles with a SPR from 500-600 nm (FIGS. 12, 13). These smaller nanoparticles also absorb laser energy, just with much less efficiency.

### DETAILED DESCRIPTION

As used herein and in the appended claims, the singular forms “a,” “and,” and “the” include plural referents unless the context clearly dictates otherwise. Thus, for example, reference to “a particle” includes a plurality of such particles and reference to “the drug” includes reference to one or more drugs, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Metal nanostructures provide catalytic, electronic, magnetic, and optical properties that are of interest in a variety of applications, including biological and therapeutic applications. The catalytic, electronic, magnetic, and optical properties of nanostructures can be controlled by geometry, size and composition. Noble metal nanostructures (including hollow nanostructure) are of interest because of their unique structural, optical properties including a tunable plasmon resonance energy absorption.

The disclosure provides a method for a simple, biocompatible route to production of monodisperse, size-controlled gold or other noble metal hollow-core nanoshells that adsorb strongly in the near infra-red. In contrast to previous techniques, the methods of the disclosure do not require a silica core particle (which may lead to problems with biocompatibility), and the nanoshells produced by the methods of the disclosure are much smaller and have smaller shell thicknesses, using less gold per particle. For example, the silica or polystyrene core assemblies are limited in size by the production of the dielectric core, which is usually at least 60-100 nm in diameter, leaving the overall core-shell particles 100-200 nm in diameter. Such particles are not easily incorporated within liposomes or vesosomes for use in drug delivery. In addition, pulsed laser irradiation of the silica core/gold shell nanoparticles causes unknown chemical reactions to degrade the silica cores, as well as melt the gold shell, which may lead to unknown toxic degradation products.

Previous techniques using dielectric core/shell structures have demonstrated potential, one major limitation is the time and labor required for fabrication. Each step leading to the core-shell nanoparticle must be accompanied by a centrifugation and rinsing step, which can reduce the yield and more importantly, destabilize the colloidal particles and lead to irreversible aggregation. Layer-by-layer self-assembly has also been used to construct core/shell composite colloidal architectures by adsorbing oppositely charged polyelectrolytes to a core particle and then adsorbing the charged gold colloids to make up the shell. For the layer-by-layer assemblies, the sizes are usually in the 1-10 micron range, which eliminates most biomedical applications. Also, in the layer-by-layer method, there is no simple way to tune the plasmon adsorption to reach the near infrared as is necessary for deep tissue penetration.

In the methods of the disclosure, the interior of the gold nanoshell is advantageously hollow and open to the surrounding fluid. Previous work on creating hollow gold nanostructures have used anisotropic silver nanoparticles as templates to generate hollow gold morphologies. However, these chemical approaches use non-aqueous solvents such as poly (vinyl pyrrolidone) in anhydrous ethanol at high temperatures that are incompatible with biomedical applications without extensive separations and purifications. Alternatively, gold nanoshells can be made via galvanic replacement at lower temperatures by employing a cobalt template, but the sensitivity to atmospheric oxygen in this process requires that the reactions be performed under a nitrogen blanket, and cobalt can be toxic and should be avoided for biomedical applications.

Hollow gold nanoshells also have significant advantages over anisotropic gold nanorods which also can be made to adsorb in the near infrared. The synthesis of the gold nanorods involves high concentrations of cetyltrimethylammonium bromide (CTAB), a cationic surfactant with membrane-compromising properties. CTAB has a poor biocompatibility profile, and CTAB-coated nanoparticles are susceptible to non-specific cell uptake, even at very low CTAB levels. Gold nanorods and other formulations containing CTAB require rigorous and expensive purification procedures prior to any biological use. Gold nanoshells also contain significantly less gold per particle than the solid gold rods, lowering the cost of the synthesis.

The synthesis methods described here are particularly straightforward and reproducible and use low temperature aqueous (“green”) chemistry and biocompatible materials to create a simple route to gold nanoshells that are about 20-100 (e.g., 20-50) nm in diameter or across for use in biomedical applications with minimal post-synthesis separations, purifications, or removal of toxic reactants.

The disclosure provides a galvanic replacement method for generating stable, tunable/scalable nanostructure including hollow nanostructure. The template replacement chemistry provided by the disclosure is rapid, stable, highly scalable, and in many cases is a true ‘one pot’ synthesis. A method of generating a porous or hollow noble metal nanostructure includes contacting a template metallic nanostructure with a noble metal salt precursor in an aqueous environment such that the noble metal salt precursor has a greater standard reduction potential than the template metallic nanostructure under conditions for galvanic replacement. For example, to create hollow metallic nanospheres (e.g., hollow gold nanospheres, HGNs) templated galvanic replacement reactions of, for example, silver for gold are used to create hollow gold nanoshells of about 20-100 nm in diameter. The metallic template core nanostructure used in the galvanic replacement are synthesized by conventional chemistries and mixed with a solution of a metallic salt to form the desired nanoshell. The metallic template core can be a silver core particle that is then oxidized to a molecular solution as the gold shell is reduced to metal. The hollow metallic nanostructure (e.g., a HGN) can be varied in size and shell thickness depending on metallic (e.g., silver/gold) reagent ratios. For example, the tuning of the hollow nanostructure can be accomplished by modifying the ratio of the metal in the metallic template nanostructure and the noble metal salt. The size and surface plasmon resonance absorbance of nanoshells made in this fashion can be tuned simply by using basic colloidal growth chemistry to control the size of the sacrificial silver sols. The template replacement chemistry is rapid, stable, highly scalable, and in many cases is a true ‘one pot’ synthesis.

Using such an approach nanostructures with a variety of shapes and geometries (e.g., spheres, triangles, cubes, rods, bowls and the like) are used as a sacrificial template to react with an aqueous noble metal salt (e.g., HAuCl₄) in a biocompatible solution stabilized by biocompatible salts such as sodium citrate. By controlling the template and the ratio of the reagents, hollow nanostructures can be obtained. Furthermore, by controlling these ratios the plasmon resonance energy of such nanostructures can be tuned by controlling the structure, thickness, composition and porosity of the resulting structure.

Suitable noble metals that can be employed include platinum, palladium, platinum-ruthenium alloys, rhodium, gold, iridium, osmium and the like. Noble metal salts are known in the art and include metals salts comprising chlorides, nitrates, acetates or others and combinations of these salts.

By the term “biocompatible anion” is meant a negatively charged counter ion which forms a salt with an ionized, positively charged group, where said negatively charged counter ion is also non-toxic and hence suitable for administration to the mammalian body, especially the human body. Examples of suitable anions include, but are not limited to, chloride ion, citrate ion, and sulfate ion. Improved stability in high ionic strength solutions can be obtained by exchanging the citrate ions for 750 molecular weight polyethylene glycol-thiol. Using these methods little or no residual chemicals from the synthesis are present that could lead to toxic problems during use or after irradiation as is the case for competing nanostructures. Typical gold nanoshells produced by this process are ˜30 nm in diameter with a ˜3 nm thick shell. The plasmon resonance can be easily tuned from 550-850 nm by controlling the size of the silver templates and the thickness of the gold shell.

For example, the core particle, which is silver in this example, is sacrificed; the gold salt that makes up the nanoshell is reduced to metal if it has a greater standard reduction potential than the template metal, which is oxidized to a molecular solution. The size and surface plasmon resonance (SPR) absorbance of nanoshells made in this fashion can be tuned very simply by applying basic colloidal growth chemistry to the sacrificial silver sols. The silver templates are first nucleated from sodium citrate and silver nitrate. The addition of sodium borohydride accelerates the chemical reactions and the resulting particles are 15-25 nm in diameter. Template particles of controlled size are grown by adding additional silver nitrate along with the “gentle” reducing agent hydroxylamine hydrochloride; this approach does not nucleate new particles. Silver is reduced onto existing nuclei and the size of the particles can be readily adjusted by controlling the added silver nitrate. The final size of the silver template controls the SPR absorbance. Without any separations needed, a controlled amount of tetrachloroauric acid is added dropwise to the solution to exchange silver for gold, with the gold depositing on the outside of the silver template as a shell. In one embodiment, the hollow nanostructures are generated in a PVP free solution. A PVP free solution is one that does not include poly(vinyl pyrrolidone).

The method of the disclosure involves the replacement reaction between a template metallic nanostructure and a noble metal salt precursor. A “template metallic nanostructure” refers to a starting nanostructure comprising any geometry (e.g., bowls, cubes, spheres, triangles, cones, tubes, wires, rings and the like). The template metallic nanostructure comprises a metal that is of a lower reduction potential than a noble metal salt precursor used in the methods described herein. Accordingly, the metal may be of any noble metal and the like.

To create metallic nanospheres (e.g., HGNs) templated galvanic replacement reactions of, for example, silver for gold are used to create hollow gold nanoshells 20-100 nm in diameter. Template core nanoparticles of a desired size are synthesized by first nucleating, then growing the templates to the desired final size, prior to mixing with a solution of a metallic salt to form the desired nanoshell. In one aspect, a silver core particle is nucleated then grown to the desired size, then oxidized to a molecular solution as the gold salt is reduced to metal on the surface of the silver template to form a hollow gold metallic shell. The metallic nanoshell (e.g., HGN) can be varied in size and shell thickness depending on the size of the template and on the metallic (e.g., silver/gold) reagent ratios. The resultant nanostructures have a hollow core. The size and surface plasmon resonance absorbance of nanoshells made in this fashion can be tuned simply by using basic colloidal growth chemistry to control the size of the sacrificial silver sols. The template replacement chemistry is rapid, stable, highly scalable, and in many cases is a true ‘one pot’ synthesis.

Nanoparticles useful in the disclosure can comprise a metal. For example, useful metallic nanoparticles comprise a metal which exhibits a low bulk resistivity. Non-limiting examples of metals for use in the disclosure include transition metals as well as main group metals such as, e.g., silver, gold, copper, nickel, cobalt, palladium, platinum, indium, tin, zinc, titanium, chromium, tantalum, tungsten, iron, rhodium, iridium, ruthenium, osmium and lead. Non-limiting examples of commonly used metals in nanoparticles include silver, gold, copper, nickel, cobalt, rhodium, palladium and platinum.

The disclosure encompasses the use of nanostructures comprising a biocompatible metallic nanostructure that has a strong radiation absorbance, in particular a strong absorbance in the wavelength range from 500-1000 nm, typically in the range from 650 nm-900 nm. Any number of metals and non-metals may be used (including alloys) that can be tuned by various geometries to obtain absorbance. The nanoparticles can comprise a single metal, such as gold, or can be layered structures, such as silica spheres covered with gold shells. When the particles comprise a single metal, gold and silver are typically used. Typically gold is used for applications related to detection in the depth of optically turbid medium, such as tissue. For gold, the plasmon resonance occurs at exceptionally long wavelengths in the near infra-red spectral range in comparison to most other metals. The disclosure also encompasses the use of nanostructures made of carbon nanotubes. Carbon nanotubes possess strong ultrasonic (pressure) response to pulsed laser irradiation in the near-infrared spectral range.

The nanostructures used in the practice of the disclosure may either be solids, that is, they can be composed of a single metal, or they can be metal shells filled with another substance. Typically the materials used have exceptionally large thermal coefficients of expansion. Examples of substances that can be contained in metal shells for the practice of the disclosure are insulators or dielectric materials such as water, gases such as nitrogen, argon, and neon, aqueous gels, such as polyacrylamide gels and gels containing gelatin, and organic substances such as ethanol. Typical fillers for metal shells are lipids, long-chain fatty acids, organic hydrocarbons, and other organic compounds comprising straight-chain hydrocarbon chains of 14 or more carbon atoms. Those skilled in the art will recognize many other substances that can be used as fillers in the practice of the disclosure.

Ligands (i.e., caps) can be linked to metallic nanostructures. Such caps can be non-functionalized, polyhomo- or polyhetero-functionalized. Nanostructures (e.g., nanospheres or nanoparticles) are capped, in one aspect, by long-chain alkyl thiols (e.g., dodecanethiol) and soluble in organic solvents (e.g., chloroform, dichloromethane, toluene, hexanes). Ligands or caps of various chemical classes are suitable for use. Ligands include, but are not limited to, alkanethiols having alkyl chain lengths of about C₁-C₃₀. In one embodiment, the alkyl chain lengths of the alkanethiols are between about C₃ to about C₁₂.

Alkanethiols suitable for use can also be polyhomofunctionalized or polyheterofunctionalized (such as, at the Ω-position, or last position of the chain). As used herein, the term “polyhomofunctionalized” means that the same chemical moiety has been used to modify the ligand at various positions within the ligand. As used herein, the term “polyheterofunctionalized” means that different chemical moieties or functional groups are used to modify the ligands at various positions. Chemical moieties suitable for functional modification include, but are not limited to, bromo, chloro, iodo, fluoro, amino, hydroxyl, thio, phosphino, alkylthio, cyano, nitro, amido, carboxyl, aryl, heterocyclyl, ferrocenyl or heteroaryl. The ligands can be attached to the central core by various methods including, but not limited to, covalent attachment, and electrostatic attachment.

In addition to alkanethiols, various suitable ligands include, but are not limited to, polymers, such as polyethylene glycol; surfactants; detergents; biomolecules, such as polysaccharides; protein complexes; polypeptides; dendrimeric materials; oligonucleotides; fluorescent moieties and radioactive groups.

Nanostructures, such as alkylthiol-capped gold colloids, are soluble or dispersible in a wide range of organic solvents having a large spectrum of polarity. Alternative capping agents, which include amines, carboxylyic acids, carboxylates and phosphines, can extend the use to virtually any solvent.

Typically synthesized nanoparticles comprising alkyl thiols are hydrophobic owing to capping with long-chain alkyl thiols. The homogeneous dispersion of these hydrophobic nanoparticles on hydrophilic oxides can therefore be a problem.

The average particle sizes and particle size distributions described herein may be measured by, e.g., SEM or TEM. Thus, the references to particle size herein refer to the primary particle size.

This method has been successfully applied to prepare gold-based hollow nanostructures and includes a wide range of different morphologies, including cubic nanoboxes, cubic nanocages, triangular nanorings, prism-shaped nanoboxes, single-walled nanotubes, and multiple-walled nanoshells or nanotubes. In addition to gold, hollow platinum and palladium nanostructures can be prepared by using appropriate salt precursors for the replacement reaction. These hollow and porous metal nanostructures show intriguing optical and mechanical properties, with their surface plasmon resonance peaks tunable from the visible to the near-infrared region. These materials are expected to find applications in a number of areas, such as optical sensing, imaging contrast enhancement, photothermal cancer treatment, and catalysis.

For example, liposome and other nanoscale carriers have been investigated for drug delivery (e.g., chemotherapy agents) to enhance efficacy and minimize toxicity by delivering more of the drug to the site of interest and minimizing systemic delivery. Large scale (entire limbs or parts of the body) hyperthermia has been used to enhance release from thermally sensitized liposomes; however, the extent of heating must be limited to minimize damage to healthy tissues. This combination of hyperthermia and drug release from liposomes has shown synergistic effects on tumor treatment. External signals have been used to induce contents release from liposomes using ultrasound, ultraviolet and visible light, and laser light, but these methods are limited to easily accessible areas such as the eye and skin, and the liposomes must be formulated from expensive or unstable lipids.

Gold nanoshells grown around silica cores have been used to selectively heat and kill tumor cells using continuous heating by NIR light. However, the maximum temperature reached by continuous heating is limited to 10-20° C. above background and the entire region to be heated must be saturated with nanoshells, which may be difficult in practice. Silica core nanoshells are difficult to synthesize relative to the hollow gold nanoshells described herein and require several steps and separations to complete. The silica/gold nanoshells are also much larger (100-200 nm diameter) compared to the 20-50 nm diameter of the HGN described here.

As described above, the methods for generating the hollow nanostructures of the disclosure provide structure having reduced toxicity and harmful agents as well as providing tunable resonance structures. Accordingly, the nanostructures obtained and provided by the disclosure find use in drug delivery compositions and methods for administration to humans and mammalian subjects. In addition, the structures can be used in research and tissue culture with reduced cytotoxicity.

Irradiation of the nanoshells with a pulsed NIR laser with sub-nanosecond pulse lengths creates high temperatures within the nanoshells that lead to collapse and annealing of the nanoshells into solid gold nanoparticles. During this heating and collapse process, the water adjacent to the nanoshells is vaporized to form unstable microbubbles which collapse and can cause lipid membranes to rupture.

The resulting nanostructures can be used in various processes and compositions directly and without further processing. However, depending upon the intended use, the structures may be decanted and resuspended in a desired buffer. For example, the tuned nanostructures can be used in drug delivery to a target site in a subject.

The disclosure provides therapeutic agent and/or diagnostic agent delivery compositions comprising a nanostructure (e.g., a nanoparticle, hollow nanosphere, nanorod and the like), a lamellar structure and an agent encapsulated within the lamellar structure. The nanostructure can be coupled to a lamellar surface, or encapsulated within the lamellar structure or in suspension with the lamellar structure. As described herein the nanostructure need not take any particular geometry, but rather should be capable of generating vibrational and/or thermal energy when contacted by electromagnetic radiation. In addition, the lamellar structure can be multivesicular liposomes, unilamellar liposomes or multilamellar liposomes. A multivesicular liposomes (MVL) refers to a man-made, lipid vesicles comprising lipid membranes enclosing multiple non-concentric chambers. A multilamellar liposomes or vesicles (MLV) refer to a composition comprising multiple concentric membranes, in between which are shell-like concentric aqueous compartments. Multilamellar liposomes and multivesicular liposomes characteristically have mean diameters in the micrometer range, usually from 0.5 to 25 μm. A unilamellar liposomes or vesicles (ULV) refer to liposomal structures having a single aqueous chamber, usually with a mean diameter range from about 20 to 500 nm.

Any liposome carrier could be modified by tethering, mixing with or encapsulating a nanostructure such as, for example, a hollow gold sphere to produce a system for rapid release on demand via NIR irradiation. Such a composition and method provides the ability to control drug delivery to selected disease sites while minimizing systemic toxicity. In addition to rapid release, only liposomes within the irradiated volume will release their contents, which provides a targeting mechanism that can localize drug release to wherever in the body that the light is focused.

This disclosure provides methods and composition useful for remote and targeted triggering of drug release from vesicle or vesosome carriers within a cell, tissue or organism, including humans. In one aspect, a vesicle or vesosome carrier is disrupted using infrared (e.g., a femtosecond pulsed near infrared (NIR) laser) irradiation. In one aspect, a hollow nanoshell is mixed with, or attached to, liposomes or vesosomes via ligand-receptor tethering, or encapsulated within lipid bilayer vesicles or vesosomes that contain a drug to be released. Nanosecond to femtosecond pulses of electromagnetic radiation (e.g., near infrared ˜800 nm wavelength) causes the nanostructure to adsorb sufficient energy to heat or to cause vibrational energy or pressure fluctuations in the surrounding media (e.g., water, buffer, or physiological fluids). The heat or pressure fluctuations cause mechanical disruption of the lipid membranes in the vesicles or vesosomes (similar to ultrasound generated cavitation or pressure fluctuations), causing an encapsulated drug or agent to be rapidly released. Both temporal and spatial control of drug release can be controlled via the application of electromagnetic irradiation external to the cell, tissue or organism.

The disclosure provides methods and composition for control of a triggered release of a drug (e.g., an intravenous chemotherapy agent in the vicinity of a tumor, an antibiotic near the site of inflammation or disease) or other agent (e.g., a diagnostic or imaging agent and the like) in which local delivery would improve efficacy, diagnosis and/or minimize side effects of treatment. Such nanostructures can also be used to simultaneously heat and/or ablate tumor tissue in combination with drug release. It also may be possible to break up kidney stones among other applications that otherwise would require ultrasound-induced cavitation.

As discussed elsewhere herein, the disclosure provides a method by which readily available hollow metallic or gold nanoshells (HGNs) are made that strongly adsorb near infrared (NIR) light. These HGN's can be entrapped within liposomes, vesicles, or vesosomes that also carry drugs. It is to be understood that the nanostructure need not be encapsulated so long as the nanostructure is within sufficient distance that upon excitation causes disruption of the lamellar vesicle. Upon excitation by a local or external radiation rapid drug release can be initiated (e.g., with NIR light pulses). Both encapsulated and un-encapsulated HGNs are capable of permeabilizing lipid bilayers and triggering the release. The compositions and methods of the disclosure can be incorporated into diverse therapeutic treatments with specific delivery of drugs and macromolecules with both spatial and temporal control using an external electromagenetic radiation (e.g. a laser source for NIR irradiation). A laser, for example, can be focused to deliver radiation only at specific sites to minimize drug delivery to healthy tissues.

The nanoparticles that are useful in metal nanoparticle compositions according to the disclosure will typically have a certain degree of purity. For example, the particles (without capping ligands) may include not more than about 1-10 atomic percent impurities, e.g., not more than about 0.1-1 atomic percent impurities, typically not more than about 0.01-0.1 atomic percent impurities. Impurities are those materials that are not intended in the final product and that adversely affect the properties of the final product.

For example, hollow gold nanoshells (HGN) thus formed can be stabilized against aggregation even in high ionic strength solutions by coating with thiolated polyethylene glycol of in the range of 750 Da-5000 Da using standard chemistry. The stabilized nanoshells can then be encapsulated within lipid bilayers. For example, the nanoshells can be encapsulated within lipid bilayers with any number of possible therapeutic or diagnostic agents. U.S. Pat. No. 6,484,889 described lamellar compositions that can be used in the methods and compositions of the disclosure as well as methods of encapsulating agents including nanostructures. In another aspect, nanostructures (e.g., nanoshells) can also be tethered to the liposome or vesosome membrane via a ligand-receptor couple such as biotin-streptavidin, or with a thiolated polyethylene glycol lipid.

Any number of different agents can be encapsulated within a lamellar structure. Various drugs including chemotherapy agents, antibiotics, antimicrobials, peptide or protein drugs, DNA or RNA, can be incorporated within vesicles by techniques available in the literature. One of the various methods of mixing, encapsulating, or tethering the gold nanoshells can be applied to any of the currently available lipid delivery methods.

The nanostructure/liposome suspension can then be irradiated, for example, with electromagnetic energy such that the nanostructure absorbs and releases vibration or thermal energy to the local environment. For example, the electromagnetic radiation can be a Ti:Sapphire laser that generates femtosecond pulses at 800 nm (or within the NIR window of 700-1000 nm) wavelength. The examples below demonstrate that such techniques provide CF dye release from the liposomes. Dye release reached a maximum 60 seconds after initiation of the laser irradiation, and a significant fraction of the dye was released within 10 seconds of irradiation. No dye was released in control systems of liposomes without hollow gold nanoshells and no dye release occurred prior to irradiation. No dye release occurred on steady irradiation with laser light at the same total energy density as the pulsed laser light.

The light pulses cause a large temperature increase in the hollow gold nanoshells, likely causing unstable microbubbles to form in the surrounding water or buffer. These unstable bubbles grow rapidly and undergo violent collapse which produces shock waves or microjets that disrupt the vesicle or liposome carriers, similar to liposome disruption by ultrasound. However, the overall energy input is such that the bulk sample temperature rises by only 1-3° C. and the only liposomes disrupted are those close to nanoshells irradiated by the laser. The nanoshells themselves are heated to melting and often collapse on themselves to form smaller solid gold particles; however, there does not appear to be significant long-term chemical or physical disruption of the liposomes or chemical changes to the dye, hence there should be no significant degradation of any drug being delivered.

The local heating and bubble formation can also be used to disrupt cells and tissues to provide a synergistic effect with the high local concentrations of chemotherapy or other drugs to provide rapid and efficient killing of cancer cells. The vesicles and nanoshells can be targeted with ligands specific for a particular type of cancer or tissue. However, the size of the vesicles and nanoshells should allow for accumulation in the tumor by the enhanced permeation and retention (EPR) effect, in which sub 500 nm particles leak through defects in the rapidly growing vasculature surrounding tumors. This combination creates the opportunity to have both spatial and temporal control of drug release via an external trigger. The potential advantages of this new photo-activated release could include (1) selective disease-cell targeting by the conjugation of accumulating particles (liposomes) and absorbing particles (HGNs) with specific antibodies, (2) localized release without harmful effects on surrounding healthy tissues, and no cytotoxicity or cutaneous photosensitivity as in photodynamic therapy, (3) deep penetration inside body with NIR light transparent to most biotissues, (4) relatively fast treatment involving short period of laser pulses. The nanoshells can also be used to provide local hyperthermia to assist in the destruction of cells as in current therapies in which gold nanoparticles are used to directly heat tissue as described below.

In accordance with the disclosure, a wavelength or wavelength spectrum of electromagnetic radiation is chosen to match the maximum of absorption for at least partially metallic spherical and non-spherical nanostructures which may be at least partially coated with organic or inorganic dielectric material or conjugated with biological molecules.

The methods of disclosure can be performed with irradiation with electromagnetic irradiation of any frequency or wavelength to cause a nanostructure to generate acoustic or pressure waves. Wavelengths in the visible or infrared range from about 200 to about 3000 nm can be used. Typically irradiation in the near-infrared wavelength range from 650 to 1200 nm is used. The irradiation can be generated with a laser, but the disclosure encompasses the use of any radiation source, regardless of whether it can be called a laser. Examples of alternative radiation sources include flash lamps, incandescent sources, klystrons, radioactive substances and the like.

An advantage of using near infrared (NIR) light to activate liposome drug release is that tissue, blood, and the like are relatively transparent to 700-1100 nm wavelength light, allowing penetration depths of about 10 centimeters. This allows sites within the body to be accessible to the NIR light to trigger drug release. Plasmon-resonant metallic nanostructures, i.e., silica core/gold nanoshells, gold nanorods, and hollow gold nanoshells (HGN) are effective at absorbing NIR light and converting this energy into heat. HGNs are similar to silica core/gold nanoshells that have been used both in vitro and in vivo to accumulate NIR light, except that HGNs have a hollow core, which allows easier synthesis and smaller overall dimensions. Gold nanoshells and nanorods illuminated with NIR light have been successfully used to non-invasively heat cells and tissues in vivo and in vitro.

In one embodiment, the vibrational and/or thermal energy generated by the nanostructures is produced through plasmon derived resonance absorption by conductive electrons in the nanostructures used in the disclosure. Suitably, the electromagnetic radiation used is pulsed and is emitted from a pulsing laser. Alternatively, the electromagnetic radiation is a modulated continuous wave.

The disclosure provides methods and compositions useful for improved therapeutic efficacy of many drugs by, for example, maximizing their concentration at the disease site; toxicity can be reduced simultaneously by lowering the concentration elsewhere in the body. Liposomes and other lipid-based drug carriers sequester toxic drugs within a lipid membrane to provide significant advantages over systemic chemotherapy by minimizing damage to healthy organs and tissues.

The disclosure provides a method by which near-complete contents release from liposomes and other lipid-based drug carriers can be initiated within seconds by irradiating a nanostructure (e.g., HGNs) with near infrared (NIR) light from a pulsed laser. For example, HGNs can be chemically tethered to the liposome surface, encapsulated within the liposomes, or even just be in solution with the liposomes. Absorbing the pulsed laser light causes, for example, HGNs to rapidly increase in temperature, leading to the formation of micro-bubbles in the vicinity of the liposome bilayer, which cause membrane rupture and content release. Only nanostructures directly irradiated are heated, thereby providing drug release localized to the volume illuminated by the pulsed laser light. Using such methods and compositions targeted release can be achieved, providing the necessary spatial and temporal control of contents release.

The methods and compositions of the disclosure can also be used to target a tissue by, for example, using a ligand targeting technique. For delivery to a desired target receptor targeted can be used. The nanostructure/liposome system presented here separates the tasks of drug retention and extended circulation to the liposome carrier, while spatial and temporal control of drug release is relegated to the nanostructure irradiated by laser light.

Alternate or additional targeting of the compositions of the disclosure can be accomplished by linking a nanostructure and/or liposome/vesosome to a species or biological or chemical substances, including for example:
(i) Antibodies and antibody fragments. These have the advantageous property of very high affinity for specific receptor sites. Both conventional and genetically engineered antibodies may be employed, the latter permitting engineering of antibodies to maximize such properties as affinity and specificity. The use of human antibodies may be used to avoid possible immune reactions against the vector molecule.
(ii) Proteins and glycoproteins other than antibodies and antibody fragments. Other useful proteins include cytokines, integrins, growth factors, cadherins, immunoglobulins, peptide hormones, lectins, selectins and pieces thereof.
(iii) Oligopeptides, polypeptides, amino acids and other protein components or protein fragments.
(v) Sugars, including monosaccharides, polysaccharides and other carbohydrates.
(vi) Vitamins, cofactors for vitamins and modified forms thereof.
(vii) Steroids, steroid analogs and modified forms thereof.
(viii) Cholesterol may be used to target endothelial cells, especially in atherosclerotic plaque.
(ix) Genetic material, including nucleosides, nucleotides, oligonucleotides, polynucleotides and modified forms of nucleosides, nucleotides, oligonucleotides, polynucleotides and other substances that bind to DNA or RNA, either through Watson-Crick pairing or through some other type of interaction.
(x) Synthetic compounds that combine a natural amino acid sequence with sequences not normally found in nature.
(xi) A completely synthetic chemical structure, that is a chemical construct not normally found in nature, with a special affinity for one or more naturally occurring receptor sites.

Those skilled in the art will recognize that many other types of targeting moieties are also possible.

The disclosure shows absorption of femtosecond pulses of NIR light induces melting and sintering of HGN tethered to, encapsulated within, or in solution with lipid vesicles containing a low molecular weight, soluble model drug, carboxyfluorescein. The high temperatures reached by the HGN induce production of unstable microbubbles, similar to the cavitation bubbles produced by ultrasound, as shown by an energy threshold for drug release and a characteristic acoustic response of the solution on irradiation. The mechanical and thermal effects of the microbubble collapse causes disruption of the liposome carriers within seconds, thereby releasing their contents as shown, for example, by an increase in the fluorescence of carboxyfluorescein entrapped with the carrier. Neither the liposomes nor the carboxyfluorescein appear to be altered during this process. The potential advantages of this new photo-activated release may include (1) synergistic disease-cell targeting by combining drug carrying particles (liposomes) and energy absorbing particles (HGNs), (2) localized release without harmful effects on surrounding healthy tissues, with no cytotoxicity or cutaneous photosensitivity as in photodynamic therapy as the gold nanoparticles are inert, (3) triggering release deep inside the body as NIR light is transparent to most tissues, (4) creating high localized concentrations of drug with both spatial and temporal control. Many liposome or polymeric carriers could be modified by tethering or encapsulating HGN to produce a system for rapid release on demand via NIR irradiation. In addition, HGN-induced liposome disruption could be used to induce rapid diffusional mixing to permit the study of fast chemical kinetics in nanoenvironments mimicking cell membranes.

The disclosure contemplates a wide variety of formulations to be used in the practice of the disclosure. In general, the nanostructure is administered simultaneously with (i.e., in the same formulation as discrete portions) or as a formulation comprising a liposome encapsulating a nanostructure or a liposome linked to a nanostructure dispersed in a sterile carrier liquid, which may be water, a water solution such as saline solution, an organic liquid, or an oil, including oils of animal, mineral or synthetic origin. The carrier may also be a mixture of several components. Examples of suitable organic liquids include, but are not limited to, methyl, ethyl, or isopropyl alcohol, acetone, glycerol, and dimethylsulfoxide. Examples of suitable oils include, but are not limited to, canola oil, soybean oil, mineral oil, and sesame oil. Water or saline solutions are typically carriers when the mode of administration is intravenous or intra-arterial injection.

The formulations used for practice of the disclosure can include a variety of excipients. The formulation may further comprise, for example, one or more chemical stabilizers, neutral lipids, charged lipids, gases, gaseous precursors, liquids, oils, diagnostic agents, and/or bioactive agents.

The compositions of the disclosure can be delivered to a subject or tissue by intramuscular, intra-arterial, intravenous, intradermal, intraperitoneal, and subcutaneous delivery. For intra-arterial or intravenous injection, the composition may be injected into the vascular system as a whole or into the vessels of a specific organ. For cancerous tumors, it will sometimes be preferable that the composition be directly injected into the tumor.

A nanostructure of the disclosure either alone or incombination with a liposomal structure can be formulated with a pharmaceutically acceptable carrier, although the nanostructure may be administered alone, as a pharmaceutical composition. Appropriate carriers and delivery methods are known in the art as described more fully herein.

A pharmaceutical composition according to the disclosure can be prepared to include a nanostructure of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “effective dose” is meant the quantity of a nanostructure, liposome or the liposomal contents according to the disclosure to sufficiently provide a desired or beneficial outcome. Amounts effective for this use will, of course, depend on the tissue and tissue depth, route of delivery and the like.

Typically, dosages used in vitro may provide useful guidance in the amounts useful for administration of the pharmaceutical composition, and animal models may be used to determine effective dosages for specific in vivo techniques. Various considerations are described, e.g., in Langer, Science, 249: 1527, (1990); Gilman et al. (eds.) (1990), each of which is herein incorporated by reference.

As used herein, “administering an effective amount” is intended to include methods of giving or applying a pharmaceutical composition of the disclosure to a subject that allow the composition to perform its intended function.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Supplementary active compounds can also be incorporated into the compositions.

The following non-limiting examples illustrate the various embodiments provided herein. Those skilled in the art will recognize many variations that are within the spirit of the subject matter provided herein and scope of the claims.

### EXAMPLES

HGNs of diameter ˜35 nm with a maximum absorption at 820 nm were synthesized via sacrificial galvanic replacement of silver core nanoparticles with tetrachloroauric acid. The HGNs were then coated with polyethylene glycol-thiol, mPEG-SH (PEG MW=750 Da) and concentrated by centrifugation. HGNs were encapsulated within dipalmitoylphosphatidylcholine (DPPC) liposomes via the interdigitated phase transition, which causes lipid membranes to form flat open sheets at low temperatures that close to form unilamellar vesicles at higher temperatures. Alternately, the nanoshells were tethered to the outside of already formed liposomes using a thiol/PEG-lipid linkage. Cryogenic transmission electron microscopy (Cryo-TEM) was used to confirm the encapsulation of HGNs inside the liposomes (FIG. 1a) or tethering to the liposomes (FIG. 1b). The HGN's could also be mixed with the liposome solution without encapsulation or tethering. 6-CarboxyFluorescein (CF) was encapsulated in the liposomes at sufficient concentration (16 mM) that its fluorescence was self-quenched. Unencapsulated CF was removed via column filtration with Sephadex G-75. In FIG. 1, the liposome membranes are almost perfectly circular in the images, consistent with a membrane under tension due to the higher osmotic pressure inside the liposome due to the encapsulated CF.

Liposome disruption was triggered by irradiating the liposome/HGNs with 800 nm NIR light from a Ti:Sapphire laser (Spectraphysics Spitfire) that generates 130 femtosecond pulses at a 1 kHz repetition rate with net power density of up to 18 W/cm². The energy incident on the samples was varied using neutral density filters. Luminescence was excited in the sample via a two-photon absorption process. The evolution of the photoluminescence was recorded with time so that the in situ fluorescence could be monitored. The CF is diluted to micromolar concentrations after release such that the fluorescence intensity is proportional to the CF concentration. Any release from the liposomes can be detected by an increase in fluorescence intensity from the background.

Irradiation with the NIR light pulses triggered a near instantaneous increase in the measured fluorescence in the solution of DPPC liposomes encapsulating HGNs and CF (FIG. 2a), but had no effect on control solutions of un-encapsulated CF, a mixture of HGNs and CF, or DPPC liposomes with CF, but no HGNs. In all samples, the CF concentrations were matched to give similar concentrations averaged over the sample volume. To relate the fluorescence intensity to the fractional release, the background fluorescence, Iₒ was subtracted from the fluorescence intensity of the solution after NIR laser treatment (Iₗₐₛₑᵣ), then divided by the maximum fluorescence, Iₘₐₓ, over background after lysis and solubilization of the liposomes by 10 molar excess of reduced Triton X-100:

$$\% release = \frac{{I}_{laser} - {I}_{o}}{{I}_{max} - {I}_{o}} .$$

To identify the mechanism of release, the laser power was varied while comparing the total fluorescence intensity after 9 minutes of irradiation. FIG. 2b shows a distinct power density threshold of NIR light to trigger release: no fluorescence was detected for a power density lower than ˜1.5 W/cm², while the total release was roughly constant at about 75% for power densities greater than 4.3 W/cm². The rate of fluorescence increase during NIR irradiation for liposomes mixed with HGNs (but not encapsulated) also increased with the laser power density (FIG. 2a). The in situ fluorescence intensity was constant for irradiation power density of 1.3 W/cm², which is below the threshold. Above the threshold, the curves could be fit with a single exponential, with a time constant increasing with decreased laser power density (time constant τ=5, 52 and 112 at the power density of 14.9, 13.0 and 7.1 W/cm², respectively). At the higher power densities, release is complete within seconds.

The power threshold suggests that the mechanism of triggered release is through perforation of lipid bilayers mediated by microbubble formation and collapse. When HGNs are irradiated by NIR light, their temperatures rise substantially; as heat is dissipated into the surrounding water, unstable microbubbles may form, grow rapidly, and then undergo a violent collapse which produces shock waves or microjets. FIG. 3a shows a typical acoustic signal of pressure fluctuations in the solution after irradiation as recorded by a hydrophone for a 0.142 mM HGN solution between two 130 fs laser pulses of power density 16.1 W/cm². The amplitude of the pressure fluctuations were at background up to a laser power density of ˜2.3 W/cm², which was similar to the power density threshold for CF dye release (FIG. 2b). The amplitude of the pressure fluctuations then increased with increased laser power density; the increased power density leads to higher HGN temperatures, which are then translated into larger pressure fluctuations in solution as this energy is dissipated. No pressure fluctuations above background were observed for control solutions of phosphate buffer solution or 4.71 μM CF solution dissolved in phosphate buffer irradiated with the highest pulsed laser power density of 16.2 W/cm².

Permeabilizing lipid membranes with micro-bubble cavitation should be induced by any HGNs in the solution, as long as the HGNs are within an optimal distance from the bilayer. To test this hypothesis, DPPC liposomes encapsulating CF dye were mixed with increasing concentrations of HGNs (FIG. 1c). Upon laser irradiation, CF release was triggered as shown in FIG. 4. The fractional CF release increases roughly linearly with external HGNs concentration (FIG. 4a) up to a HGN concentration of 0.0315 mM (at higher HGN concentrations, the CF fluorescence is quenched by the gold). To minimize the distance between the HGN and the lipid bilayer, HGNs can be conjugated directly to the liposome bilayer via a thiol-PEG-lipid linker (FIG. 1b). Tethering the HGN directly to the outer surface of the liposomes increases the maximum release fraction to 96% (FIG. 4b). The efficiency of photo-triggered contents release is strongly affected by the proximity of HGN to the bilayer, consistent with the hypothesis that mechanical disruption by microbubbles is responsible for release.

In addition to inducing liposome contents release, cryo-TEM images of irradiated samples show that the morphology of the HGNs has changed after irradiation; the hollow center of the nanoshell has collapsed consistent with a significant increase in the HGN temperature (FIG. 5). The changes in morphology are also shown by UV-VIS spectroscopy; the 820 nm absorption peak of HGNs gradually disappears with irradiation, along with the growth of a peak at ˜530 nm, which is a typical of solid gold nanoparticles. The HGNs reach sufficiently high temperatures after femtosecond pulses of NIR light to melt and anneal into more stable shapes; dissipation of heat to the surrounding water is slower than the electron dynamics involved in plasmon-mediated heating. The high temperature gradients around the gold nanoshells can cause the formation of unstable microbubbles as the heat is dissipated to the water (30 nm diameter particles thermally equilibrate with their surroundings in microseconds), and hence give rise to the mechanical and thermal effects associated with ultrasound induced cavitation. Even though the gold nanoshells are heated to melting, the temperature increase of the bulk solution was less than 1° C. above ambient; CF release was not due to changes in the permeability at the 41° C. phase transition temperature of the DPPC liposomes. Minor differences in the liposome morphology are visible after irradiation; the bilayers are less circular, the bilayer appears to be under less tension, consistent with the decrease in the positive osmotic pressure difference caused by CF release. Continuous (unpulsed) laser irradiation at 800 nm with the same averaged power density led to no increase in the fluorescence intensity, and hence no CF release, even after 4 hours (FIG. 4b). When continuous laser irradiation is used, the nanoshell is always close to being in thermal equilibrium with its surroundings and there are insufficient temperature gradients to give rise to microbubble formation. This is an important difference with continuous wave heating of tumors with silica/gold core/shell nanoparticles; the light adsorption is used as a low temperature source of energy to heat the volume around the nanoparticles to 10-20 C above ambient temperature. No large temperature gradients are generated.

The observations of (1) a strong dependence of dye release on HGN concentration and proximity to the liposome bilayer, (2) a power density threshold for liposome contents release; (3) a similar threshold for the acoustic signature of pressure fluctuations in solution; and (4) melting and annealing the HGNs in solution, confirm the hypothesis that liposome disruption is due to mechanical deformation and perforation of the lipid membrane induced by unstable, cavitation-like microbubbles.

Synthesis of hollow gold nanoshells. Silver seed nanoparticles were prepared by reducing a well-stirred solution of 600 mL 0.2 mM AgNO₃ with 0.6 mL 1.0 M NaBH4 in the presence of 0.5 mM sodium citrate. The solution was stirred for at least half an hour to allow NaBH4 to fully hydrolyze. Larger silver nanoparticles to be used as sacrificial templates for the gold nanoshells were grown from the silver seed solution by adding 0.6 ml of 2.0 M hydroxylamine hydrochloride and 1.5 ml 0.1 M AgNO₃ and stirring overnight. The HGN were formed via sacrificial galvanic replacement of silver with gold by quickly mixing the silver nanoparticles with 3.8 ml of 25 mM HAuCl4 solution at 60 C. The silver template is oxidized to a molecular solution as the gold is reduced to metal, which deposits on the original silver nanoparticle template, resulting in a hollow gold nanoshell. The size of the nanoshells were directly related to the size of the sacrificial silver templates. The nanoshells were spherical in shape and monodisperse in both overall diameter and shell thickness.

The HGNs can be further sterically stabilized against aggregation by reacting the HGN with polyethylene glycol via a Au—SH linkage. The m750PEG-SH was prepared by mixing methoxypolyethylene glycol amine (mPEG-NH2, molecular weight of the PEG polymer segment is 750 Da, Sigma Aldrich, St. Louis, Mo.) and 2-imminothiolane in aqueous buffer (2.28 mM Na2HPO4, pH 8.8) at a final concentration of 0.0379 mM and 0.078 mM, respectively. 0.5 ml of the as prepared 0.0379 mM m750PEG-SH was added to 600 ml HGN to achieve a 1000:1 ratio of thiol: gold. The PEG-HGN solution was centrifuged and dispersed in milli-Q water twice (21000 g, 30 min per cycle) to remove soluble inorganic species. The pellet readily re-disperses in water or buffer. The UV-VIS absorption spectra of HGN were recorded on a Jasco V-530 spectrometer (JASCO Corp., Tokyo) and showed a surface plasmon resonance peak at 820 nm (FIG. 1D).

Encapsulation of HGN and CF inside liposomes. 6-CarboxyFluorescein (CF, Invitrogen, Eugene, Oreg.) was dissolved in water together with 6 equivalents of concentrated NaOH, which converts the CF from its acid form to the water-soluble salt form. The CF solution described was used to disperse the HGN pellet described above. Dipalmitoylphosphatidylcholine (DPPC) was purchased from Avanti Polar Lipids (Alabaster, Ala.) and was used as received. The dry lipid was hydrated by Milli-Q water and vortexed at 55° C. to disperse and hydrate the lipid. DPPC unilamellar vesicles (50 nm in diameter) were prepared by sonication at room temperature using a 60 Sonic Dismembrator (Fisher Scientific, Atlanta, Ga., USA) for 4 minutes at a power of 4 W. The transition from the normal (L-alpha) bilayer phase to the interdigitated (LI) bilayer phase was induced by the addition of 0.106 mL of ethanol (3 M net ethanol concentration) to 0.5 mL of a 50 mg/mL DPPC vesicle suspension. The initially bluish vesicle suspension turned milky white, and its viscosity increased significantly. After sitting at 4° C. for overnight, the interdigitated sheets were centrifuged and dispersed in milli-Q water 3 times to remove ethanol. With all the supernatant removed, the pellet of interdigitated DPPC sheets were mixed with the solution of CF and HGN. The mixture was then heated at 50° C.—above the 41° C. main transition temperature of DPPC—for 2 hours under vortex mixing, driving the sheets to close around the HGN in suspension to form the interdigitation-fusion vesicles. The formed vesicles were passed through a polycarbonate filter with a pore size of 400 nm with the aid of a lipid mini-extruder (Avanti Polar Lipids Inc., Alabaster, Ala.). Mixing of the CF and HGN with the pellet of DPPC interdigitated sheets gives final concentrations of 16 mM CF (110 mOsM), 12 mM HGN and 22 mg/ml DPPC. About 50-60% of the HGN is expected to be encapsulated in liposomes.

FIG. 8 shows a tilt series of cryo-TEM images that show the HGN are encapsulated within the liposomes after this procedure. After the encapsulation step, buffer (denoted as external buffer: 16.8 mM Na HPO₄, 3.2 mM NaH PO₄ and 34.5 mM NaCl, pH=7.4) was used to disperse the DPPC liposomes containing CF and HGN to minimize osmotic stress across the membrane. The unencapsulated CF was removed by size-exclusion chromatography using a Sephadex G-75 column (Amersham Biosciences Corp., Piscataway, N.J.) eluted with the external buffer. The eluted suspension was twice centrifuged at 100 g for 30 minutes and re-dispersed in the external buffer of DPPC vesicles to remove any unencapsulated HGN.

Tethering HGN to liposomes containing CF. The HGNs were tethered through Au—SH-PEG lipid linkages to modified DPPC liposomes with CF encapsulated inside. First, the pellet of interdigitated DPPC sheets was prepared by adding ethanol to 50 nm DPPC liposomes and then centrifuged to remove ethanol. The interdigitated sheets were modified by mixing CF solution along with the amine-modified phospholipid distearoylphosphoethanolamine-amino (polyethylene glycol) 2000 (DSPE-2000PEG-NH₂, Avanti Polar Lipids, Alabaster, Ala.) as a dried powder to give 2 mole % of the DSPE-2000PEG-NH₂ in the bilayer. The mixture was then heated at 50° C. for 1 hour under vortex mixing, driving DSPE-2000PEG-NH2 to be incorporated into DPPC sheets as the sheets closed and formed the interdigitation fusion vesicles. Next, the amine groups at the liposome surfaces were converted to thiol by mixing with 100% excess 2-iminothiolane solution. The DPPC/2 mole % DSPE-2000PEG-SH liposomes encapsulating CF were incubated with a solution of HGN and CF for 48 hours to allow HGN to tether to the outer surfaces of liposomes (See FIG. 9). The final concentrations in the solution were 18 mg/ml phospholipid (98 mole % DPPC and 2 mole % DSPE-2000PEG-SH), 18 mM HGN and 16 mM CF. The liposomes with tethered HGNs were eluted through a Sephadex G-75 size-exclusion column and centrifuged to remove unencapsulated CF and free HGNs.

Mixing liposomes containing CF with external HGNs. DPPC liposomes containing CF were prepared by the same interdigitation-fusion method described above except no HGNs were added. In brief, CF solution in external buffer was mixed with the pellet of interdigitated DPPC sheets, and then heated to 50° C. under vortex mixing to form interdigitation fusion vesicles. The formed vesicles were extruded through a 400 nm polycarbonate filter and then eluted through a Sephadex G-75 column to remove external CF.

The internal CF concentration was 16 mM. A pellet of PEG-HGNs concentrated by centrifugation at 21000 g was dispersed in the external buffer, and then added to the pre-formed DPPC liposomes at different concentrations.

Pulsed laser irradiation. The samples were irradiated by the output of the femtosecond (fs) Ti: Sapphire regenerative amplifier (Spectraphysics Spitfire) running with 1 kHz repetition rate. The laser beam was collimated by a Galilean telescope to achieve a Gaussian diameter of 2.3 mm. Pulse duration was monitored by a home-built single-shot optical autocorrelator and was kept at about 130 fs. The spectral FWHM of the laser radiation was ˜12 nm centered around 800 nm. The laser beam was directed onto the sample by a system of mirrors, no focusing optics were used. The energy of the optical pulse was controlled by Schott neutral density glass filters. A thermopile power meter (Newport Inc., Santa Ana, Calif.) was used to measure the incident optical power. The maximum power available in experiments was 670 mW, which corresponds to 670 μJ/pulse and a power density of ˜16 W/cm².

Luminescence was excited in the sample via two-photon absorption process. The emission was collected at a 90 degree angle by a system of lenses and focused on the entrance slit of a monochromator (Acton Research SpectraPro 300). The laser radiation was blocked by a Schott colored glass filter (BG38). The light dispersed by the monochromator was detected by a spectroscopic CCD camera (PI Acton PIXIS-400) and transferred into a PC. The evolution of the photoluminescence was recorded by collecting consecutive spectra over a 600 nm bandwidth with a constant interval. The solution was stirred to ensure good mixing of the sample during irradiation. Any release of CF from the vesicles was detected by an increase in fluorescence intensity from the background fluorescence level as the external concentration of CF increased. Fluorescence was measured using a Perkin Elmer LS-55 fluorometer equipped with a xenon lamp excitation source and a Hamamatsu (Japan) 928 PMT, using 90 degree angle detection for solution samples.

Cryo-Transmission Electron Microscopy (Cryo-TEM). Thin sample films were prepared under controlled temperature and humidity conditions within a VitRobot (FEI Company, Oregon), then vitrified by rapid plunging into liquid ethane. Cryo-TEM was performed on a JEOL 2010 microscope operating at 200 kV with a Gatan liquid nitrogen specimen cryo-holder. A series of images with different degree of tilt for 3-D reconstruction were obtained with a high angle tilt cryogenic holder at tilts of up to ˜70°.

Hydrophone measurements of pressure fluctuations. The pressure fluctuations in samples undergoing laser irradiation were measured using a hydrophone (model TC4013, Reson, Goleta, Calif.). The bandwidth of the hydrophone is 1 Hz-170 kHz (−10 dB). The hydrophone diameter is 0.5 cm and the length is ˜2 cm. The hydrophone was mounted in the quartz cuvette with a 10 mm light path and 3.0 ml capacity. The hydrophone was submerged under the solution and placed ˜5 mm above the laser path and the cuvette was filled with 2.5 ml solution. The cuvette was mounted in the path of the pulsed fs Ti: Sapphire laser and the solution inside the cuvette was not stirred during the laser irradiation.

The output of the hydrophone was recorded with a storage oscilloscope. The data collection was triggered by the laser pulse. A typical resultant waveform is shown in FIG. 3a. The energy of the laser pulse was controlled by Schott neutral density glass filters. Each measurement was summarized by the standard deviation of the amplitude of the pressure fluctuation signal collected by the hydrophone as in FIG. 3b.

Nanoshell synthesis and characterization. Gold nanoshells were prepared by reducing gold tetrachloroauric acid (HAuCl₄) onto silver nanoparticle templates.

Silver nanoparticles were prepared at 60° C. in a well stirred solution of 50 mL 0.2 mM silver nitrate (AgNO₃, Aldrich) in the presence of 0.5 mM sodium citrate following a 1 mL 100 mM sodium borohydride (NaBH₄, Aldrich) injection (producing a characteristic yellow color). The silver sols were allowed to stir at 60° C. for a minimum of two hours. After cooling to room temperature, larger silver nanoparticles could be grown from these stock sols, if desired. Silver particle growth was initiated by adding 1 mL of 200 mM hydroxylamine hydrochloride (NH₂OH.HCl, Aldrich) to the silver sol followed by stirring for five minutes, followed by addition of 0-1 mL of 0.1 M AgNO₃. The growing silver nanoparticles turned the sol a darker yellow or orange depending on the amount of additional AgNO₃. After stirring for a minimum of two hours (and often aging overnight), gold nanoshells were made via galvanic replacement chemistry from the template silver sols. First, 50 mL of a given silver sol was heated to 60° C. and the necessary amount of 25 mM tetrachloroauric acid (HAuCl₄, Aldrich) was added dropwise (depending on the initial silver template size). The reactions were monitored using UV/vis/NIR spectroscopy, and stopped when the silver peak located near 400 nm vanished, which occurred when the gold/silver ratio in the reaction vessel approached the stoichiometric ratio of 1:3 (to err on the side of completion, the ratio was usually 0.37:1). Once the reaction was complete, the samples were cooled, silver chloride was allowed to precipitate, and the supernatant containing the gold nanoshells was transferred to another vessel and stored at 4° C. until further use. Nanoshells prepared in this fashion are stabilized by biocompatible citrate anions and were stable against aggregation for months.

Nanoparticle concentrations were ˜10¹⁰-10¹¹/mL, based on the average dimensions measured using transmission electron microscopy (TEM) and the known amounts of materials used during the reaction. For TEM nanoshell suspensions were spread onto formvar-coated TEM grids (Ted Pella, Redding, Calif.) and dried in vacuum. Digital TEM micrographs were obtained using an FEI Technai Sphera TEM (FEI, Netherlands) equipped with a CCD camera (Gatan, Pleasanton, Calif.) operating in transmission mode at 200 keV. X-ray photoelectron spectroscopy (XPS) using a Axis instrument was used to map the relative composition of nanoshells after galvanic replacement chemistry.

Laser irradiation. The particle suspensions were irradiated with a Ti:Sapphire laser (Spectraphysics Spitfire) that generates 90 fs pulses at a 1 kHz repetition rate with energies up to 800 μJ/pulse. The energy incident on the samples was varied using neutral density filters. The Gaussian width of the beam was 2.3 mm. Typically, 3 mL of the sample was irradiated in a quartz cell with a 1-cm path length. Samples were irradiated for 10-15 minutes, and the transmitted intensity was measured with a power meter. This irradiation time was sufficient for the measured transmitted intensity to reach a steady value. A Teflon stir-disk was placed at the bottom of the cell to ensure good mixing. The cell was placed atop a magnetic stir-plate.

Calorimetry. The temperature of the particle suspensions in the irradiation cell was measured using a 0.0625 inch thick stainless steel K-type thermocouple probe (Omega Engineering, Inc., Stamford, Conn.) inserted through the plastic cap into the top few millimeters of fluid (well away from the laser path). The thermocouple was linked to an Omegaette HH306 digital thermometer (Omega) that was interfaced to a computer using Thermolog data acquisition software (Omega). The extinction (absorbance) spectra of the samples before and after irradiation were measured with the UV-vis spectrophotometer. After irradiation, each sample was imaged with TEM.

The method provides a simple and reproducible route to gold nanoshells 20-50 nm in diameter. Template silver nanoparticles of a desired size are synthesized and then mixed with a solution of a gold salt that forms the desired nanoshell. However, unlike many of the approaches mentioned above, the core particle is sacrificed and the nanoshells are hollow; the metal salt that makes up the nanoshell is reduced to metal if it has a greater standard reduction potential than the template metal, which is oxidized to a molecular solution. Gold has a relatively large standard redox potential (0.99 V vs. SHE), and can be reduced by a variety of metals having lower potentials such as silver or cobalt. The reduced zero-valence gold metal nucleates near the dissolving template core, which acts as the electrode surface, and the resulting shell eventually becomes complete (although residual pinholes or defects often remain). The resulting particles can be varied in size and shell thickness depending on reagent ratios and have a hollow core that can be filled with the suspending solution or other materials. Here, silver template particles are used as any residual Ag⁺ ions could be retained in the sol as an additional biocide since Ag⁺ is known for its antimicrobial properties. For imaging and sensing purposes, the hollow core of the shells makes their surface plasmon resonance (SPR) more sensitive to their surroundings.

The size and surface plasmon resonance (SPR) absorbance of nanoshells made in this fashion can be tuned very simply by a secondary, gentle reduction with silver nitrate to grow the silver particle nuclei to the desired size. The emphasis here is to create a simple and scalable route to gold nanoshells for practical applications with tunable sizes and absorbance profiles that require minimal experimental footprints (reduced heating, etc.), only aqueous solvents, low temperatures and biocompatible materials. The template replacement chemistry is rapid, stable, highly scalable, and in many cases is a true ‘one pot’ synthesis without many of the inconvenient steps.

Femtosecond light pulses in the NIR can heat these nanoshells sufficiently to melt and/or ablate the nanoshells into smaller nanoparticles, with an accompanying shift in the plasmon absorbance. These localized temperature pulses may be useful to triggering drug release, or creating nanometer scale hot-spots that could lead to cell death. The more unexpected result is that the maximum temperature of the individual nanoshells is governed by a competition between the red-shift of the SPR peak (which increases the overlap with the NIR irradiation), which increases with the nanoshell diameter to shell thickness ratio and the increased mass of the nanoshells (which increases roughly as the square of the nanoshell diameter). Optimizing the nanoshell structure for maximum light absorption in the NIR does not necessarily provide the highest maximum temperatures for the individual nanoshells during pulsed irradiation; however, it is not yet known which is most important for biomedical applications such as photothermal destruction of cancer cells or drug release from liposomes.

Stable suspensions of silver nanoparticles to be used as a nuclei for templates were synthesized in minutes using sodium citrate and silver nitrate. The addition of sodium borohydride accelerates the chemical reactions, and the resulting nuclei are ˜15-25 nm in diameter. Larger silver particles for use as templates can be formed from these nuclei by reducing additional silver nitrate in the presence of hydroxylamine hydrochloride. Hydroxylamine hydrochloride is a ‘gentle’ reducing agent, and does not induce secondary nucleation of new silver particles. FIG. 10a shows that the total absorbance at the 400 nm peak associated with the plasmon resonance of silver nanoparticles increased with increased silver nitrate concentration. There was a slight red-shift in the SPR peak as the particles grew, but was much less pronounced than that observed for gold nanoparticles (all suspensions were diluted ten-fold prior to spectrophotometry).

FIG. 10b shows that on a log-log scale, that the average hydrodynamic radius measured by dynamic light scattering (DLS) increased linearly from the initial 28 nm of the original 0.2 mM stock sol with increasing silver nitrate concentration. The ⅓ slope of the line through the data, (the origin of which was set to the initial average diameter of 28 nm as measured by DLS), consistent with the bulk of the added silver being added to existing nanoparticles with a minimum of new nucleation as given by a simple mass balance:

$$\frac{{D}_{f}}{{D}_{i}} = {( \frac{{A}_{n} + {A}_{g}}{{A}_{n}} )}^{1 / 3} \qquad ( 1 )$$

in which D_{f} is the final nanoparticle diameter, Dₙ is the diameter of the initial silver nuclei, Aₙ is the silver nitrate concentration needed to grow the nuclei, and A_{g} is the added silver nitrate needed to grow the particles. Hence, the increase in absorbance in FIG. 10a is due to the increase in total silver concentration at a constant overall particle concentration. Control over the silver template size distribution was essential to controlling the SPR absorption peak of the gold nanoshells.

The silver nanoparticles were converted into gold nanoshells by adding HAuCl₄ directly to the as-grown nanoparticle solution, with no centrifugation or rinsing steps. This greatly simplifies the overall production of nanoshells and prevents any irreversible aggregation of the particles that often accompanies centrifugation or other purification methods. Silver (Ag⁺/Ag 0.8V, vs SHE) has a lower redox potential than does gold (AuCl₄⁻/Au 0.99V, vs SHE) and the replacement reaction is:

3Ag₍ₛ₎+AuCl_{4(aq)}⁻→Au₍ₛ₎+3Ag_{(aq)}⁺+4Cl_{(aq)}⁻

Three silver atoms must be oxidized for every gold atom reduced. Upon the addition of the concentrated HAuCl₄, the solution turns from yellow/orange to grey/yellow to blue/grey to blue/turquoise within seconds as the silver and gold were oxidized and reduced, respectively. The process was complete in a few minutes according to the UV/vis spectra, but the reaction mixture was stirred for an additional hour, then refrigerated at 4° C.

FIG. 11a shows the evolution of the UV-NIR spectra as different amounts of HAuCl₄ were added to the silver nanoparticles grown from the original 0.2 mM AgNO₃ stock sol. The gold peak gradually red-shifts during the reaction to about 620 nm from 400 nm. This is consistent with independent nucleation and growth of the gold nanoshells around the dissolving silver template until the shell knits together and is complete, giving the final surface plasmon resonance. However, the point where the silver peak and the gold peak cross is at nearly the same wavelength and intensity throughout the reaction. This transition point is referred to as an isosbestic point and is indicative of a direct transition between two states (in this case between metallic silver and gold). If there were intermediate states, these would manifest themselves as additional peaks between the initial and final peaks. Regardless of the initial silver template size, gold nanoshell formation is direct and there is little secondary nucleation of gold nanoparticles, otherwise an SPR peak located near 520 nm (indicative of solid gold nanoparticles) would appear. The final ratio of added gold to silver was always slightly greater than ⅓ to insure that the reaction went to completion (0.37-0.38:1).

Increasing amounts of gold were required to form the shells around the larger silver template cores. The same 0.2 mM stock silver sol was used in all cases (10¹⁰-10¹¹ particles/mL), so the particle concentration is approximately the same regardless of the final silver nanoparticle size. The magnitude of the gold surface plasmon resonance is concentration dependent (FIG. 11b), and follows a shift to longer wavelengths, which is expected from nanoshells with roughly constant wall thickness, but increasing diameter as predicted by Mie theory calculations of the extinction behavior (FIG. 11c). The wavelength of the SPR increases with the initial silver concentration (which is also a measure of the initial particle size or relative ratio of shell to diameter, See FIG. 10b), providing a rough guide to tuning the SPR absorbance maximum across the NIR region (FIG. 11d). A macroscopic view of the gold nanoshells (some which have spectra plotted in FIG. 11b) is shown in FIG. 11e demonstrating the characteristic change in color with increasing silver template size. These characteristic colors make it simple to distinguish between nanoshells as a function of size and shell thickness.

X-ray photoelectron spectroscopy (XPS) verified that silver was present with the gold metal after the galvanic replacement took place, but not the chemical state, Ag⁺¹ or Ag⁰, of the silver. The silver signature was present even after the samples had been dialyzed in 5 mM citrate solutions for several days, indicating that the silver was insoluble and associated with the nanoshells. XPS provides a means for mapping the chemical composition of the first several nanometers of a surface. Thus, it is not surprising that silver was detected via XPS as silver and gold have been shown to alloy on the colloidal scale, and the initial ratio of silver to gold in any suspension is 3:1. However, the hollow structure shown in the TEM images and the absence of silver resonances in the UV/vis spectra indicate that very little metallic silver remains. The electron densities of both gold and silver are sufficiently high to render any silver/gold core/shell particles more than 20 nm in diameter opaque in a TEM image. Opaque particles were not observed with TEM, thus very little metallic silver remained after the galvanic replacement reaction. For these purposes, attaining tunable SPR frequencies in NIR window for sub 100 nm gold particles is sufficient; any residual silver will not negatively impact applications. In fact, Ag⁰ or Ag⁺¹ have antimicrobial properties and their presence may provide additional benefits to the sol.

The SPR peak of the gold nanoshells can be readily tuned from 600-800 nm simply by adjusting the amount of silver present in the silver growth sol.

The effect of NIR laser irradiation upon hollow gold nanoshells. The citrate and sodium borohydride reduced (golden yellow colored) silver nanoparticles used for these studies were stable for months. It is well-established that silver and gold nanoparticles made via similar chemistries are stable against aggregation for years. The particles are stabilized against flocculation by residual ions adsorbed to the particle surfaces (e.g. citrate and or BO₂⁻ ions), imparting a net negative surface charge. The gold nanoshells made via silver template particles were stable for many weeks or months, thus it is likely that they have also adsorbed charged species that provide electrostatic stability. The surface charge of these particles is negative, as they tightly adhere to positively charged surfaces. If any settling did occur, brief sonication was sufficient to re-suspend the particles.

The gold nanoshell suspensions remain their original color (the actual color varied with composition or size of the particles, See FIG. 11e) after short periods of irradiation at 800 nm. However, with increased total energy input, the gold nanoparticle sols eventually turned reddish in color, indicating a change in the particle morphology. After irradiation, the nanoshell suspensions were less stable, settling occurred within 24 hours of irradiation (regardless of the energy level used). Particles could be re-suspended by sonication, but several repetitions of settling and re-dispersing revealed the onset of visible dark flocs (indicating irreversible aggregation).

After irradiation, the peak maximum settles between 500-550 nm, consistent with the expected absorbance of 10-40 nm solid, spherical nanoparticles (See TEM images in FIG. 13).

The spectra of the larger particles (FIG. 12c, d) first exhibited a red-shift to longer wavelengths upon irradiation at low intensity; new peaks appeared at longer wavelengths (˜900 nm) that had lower magnitudes than the original SPR peak (indicating lower concentrations). Prior to irradiation, these larger gold nanoshells were more polydisperse, and exhibited broader SPR peaks than their smaller counterparts (See FIGS. 11b, c). Eventually, the SPR peak shifted to wavelengths between 500-600 nm at maximum laser power as in FIGS. 12a, b (˜700 μJ/pulse); again indicating that smaller spherical particles were being formed due to the plasmon heating. This process is sometimes referred to as ‘hole burning,’ in which a portion of an absorption peak is removed via direct irradiation at that wavelength. The broad SPR of the un-irradiated sols is likely a summation of the different resonances of nanoshells that are polydisperse in both shape and size (see FIGS. 6, 14). However, the SPR peak near 800 nm decreased rapidly upon laser irradiation (hole burning) as those particles responsible for the 800 nm peak melted and were reduced in size, revealing other absorbance peaks that were present before irradiation (at lower intensities, due to the lower concentration of nanoparticles with resonances at these other wavelengths). The apparent transition to longer wavelengths has been observed in other systems including gold nanorods, which can have an axial SPR in the NIR window. As the laser energies increase, all particle populations melt and shift their SPR to 500-600 nm consistent with the transition to solid spherical nanoparticles.

FIG. 13 shows TEM images of the evolution of the nanoshells after 10 minutes of irradiation with 800 nm light pulses of either 350 or 700 μJ energy. After irradiation at 700 μJ, all of the nanoshells, regardless of initial size, appear to have sintered and annealed into solid, spherical particles. There was a collapse of the hollow core, sometimes leaving a small residual hollow within the melted particle. However, images at 350 μJ show that instead of simply collapsing to form smaller, solid spherical nanoparticles, the larger nanoshells sometimes break apart upon absorbing the photon energy, leading to a disperse collection of asymmetric incomplete shells, oblate spheroids, rods and branched structures such that several different SPR resonances likely result. The elimination of the 800 nm peak seen in FIGS. 12c,d may be the result of these changes in particle shapes. However, the images of the nanoshells exposed to the highest laser power show that all the nanoshells examined reach the melting point of gold (>1064° C.) and eventually condense into solid spherical nanoparticles of 10-40 nm diameter.

FIG. 14 shows the absorption kinetics of the nanoshells which were followed by measuring the light transmitted through the sample; whatever light is not transmitted is absorbed by the nanoshells or scattered (see FIG. 15). In FIG. 14a, irradiation of 20 nm diameter nanoshells (0.098 mM Au) with a SPR ˜600 nm show a rapid increase from about 50% transmission to 75-80% transmission within 1 minute for all of the incident light pulse energies. The transmitted energy is then almost constant suggesting that the particles are no longer changing shape, which means that the absorption due to the SPR is constant as well.

In FIG. 14c, for 40 nm diameter nanoshells (˜0.25 mM Au) with an SPR closer to the 800 nm wavelength of the laser (See FIG. 11b, 12), the fraction of laser power transmitted through the sample depends much more on the pulse energy. Consistent with the SPR peak overlapping the incident light wavelength, the initial transmission was close to zero. For a pulse energy of 161 μJ, the transmitted intensity increases only to about 25 μJ after 10 minutes; only 16% of the incident energy was transmitted. In comparison, for a pulse energy of 700 μJ, 410 μJ or ˜60% of the incident energy was transmitted after 10 minutes. These differences in transmitted intensity with time are likely due to different changes in the nanoshell morphology (FIG. 13) that lead to the changes in nanoshell absorption as shown in FIG. 12. With increasing laser pulse energy, FIG. 13 shows that all of the nanoshells eventually melt and anneal into more stable solid nanoparticles with less adsorption at 800 nm. For smaller irradiation energy, the larger nanoshells break up to form shapes with new absorption peaks that still absorb a substantial fraction of the incident light. The larger particles apparently take more time to anneal into the final spherical gold particles that are the stable form after irradiation. FIG. 14d summarizes the kinetics of the change in transmission for the highest pulse energy of 680 μJ with the gold concentration, and hence the size and SPR of the gold nanoshells. The largest particles anneal more slowly than the smaller particles, even though the nanoshell SPR peak overlaps more with the incident 800 nm wavelength light. Only 44% of the incident light is transmitted through the sample with the 50 nm nanoshells (0.42 mM Au) compared to the 81% transmission through the 20 nm nanoshells (0.098 mM Au) after 10 minutes of irradiation.

The kinetics suggest that the smaller nanoshells anneal more quickly than do the larger nanoshells, even though more total energy is absorbed by the larger nanoshells. From the known particle absorbance and concentration, a simple energy balance incorporating Beer's law can be used to determine the energy per gram of gold, Q (J/g), that the nanoshells of various sizes absorb during a given NIR laser pulse:

$$Q = \frac{{{I}_{0} ( 1 - ξ )}^{2} ( 1 - {10}^{- A} ) {ɛ}_{A}}{[ Au ] σ l} \qquad ( 2 )$$

A is the absorbance (−log₁₀ of the transmitted light), [Au] is the nanoshell gold mass concentration in the sol, σ is the Gaussian beam area (0.042 cm²), l is the laser path length through the cuvette (1 cm), I₀(1−ξ)² is the incident laser intensity adjusted for reflectance losses from the cuvette/air interfaces ((1−ξ)²˜0.9 for all of the cuvettes used), and ε_{A} is the relative absorption efficiency of the gold nanoshells (a measure of how much of the non-transmitted light is absorbed rather than scattered). The energy absorbed per nanoshell per pulse is Q_{abs}=ρ_{g}VQ, in which ρ_{g} is the density of gold (19.32 g/cm³) and V is the volume of gold in the nanoshell (in cm³). FIG. 15 shows Mie calculations for the extinction, scattering and adsorption efficiencies for 30 and 50 nm diameter cores with 7 nm thick shells. The ratio of the absorption efficiency to the extinction efficiency gives an estimate of the relative absorption efficiency, ε_{A}. ε_{A}˜0.95 for 30 nm diameter nanoshells with a 7 nm wall thickness and ε_{A} decreases to ˜0.8 for 50 nm diameter nanoshells with a 7 nm wall thickness. As the particle sizes increases, scattering increases and the relative absorption efficiency decreases, although this is partially compensated by the stronger overlap of the larger nanoshells with the irradiation laser wavelength of 800 nm.

From the spectroscopy and TEM images, it is apparent that the shape and sizes of the nanoshells are significantly altered by irradiation, implying a large rise in nanoshell temperature. From the estimate of the energy absorbed per particle from Eqn. 2, the temperature reached by the gold nanoshells during a light pulse can be calculated if heat transfer to the surrounding solution is slow in comparison to the plasmonic heating. To determine the temperatures reached by the nanoshells, it is necessary to consider if the irradiation energy per gram, Q, is sufficient to heat the nanoshell to the melting temperature of gold, Tₘ (1064° C. is the bulk melting point of gold). For nanometer dimension particles, the melting temperature of spherical gold particles decreases with decreasing size, but the effect is minimal for particles greater than about 20 nm in diameter. If, Q≦C_{pg}(Tₘ−Tᵢ) (C_{pg} is the heat capacity of gold (0.129 J/g−K), taken to be constant for liquid or solid phases and Tᵢ is the initial temperature of the nanoshell, ˜135 J/g is required to reach the melting point of gold from 20° C.), then

T=Tᵢ+Q/C_{pg}. (3a)

If the nanoshell reaches the gold melting temperature (which may be slightly suppressed due to the nanometer scale dimensions of the particles), the enthalpy associated with melting the gold, ΔH_{Melt} (64 J/g) must be supplied before any further increase in temperature occurs: that is, T=Tₘ for

$${C}_{pg} ( {T}_{m} - {T}_{i} ) < Q < {C}_{pg} ( {T}_{m} - {T}_{i} ) + Δ {H}_{melt} ; \\ 135 \frac{J}{g} < Q < 199 \frac{J}{g} \qquad ( 3 b )$$

For Q>199 J/g:

$$T = {T}_{i} + \frac{Q - Δ {H}_{melt}}{{C}_{pg}} \qquad ( 3 c )$$

Finally, if the temperature of vaporization of gold is reached (2856° C., Q>430 J/g), then the enthalpy associated with gold vaporization, ΔH_{Vap}, (1870 J/g) must be supplied before any further increase in temperature occurs.

From Eqn. 2, the energy associated with light adsorption can be estimated and set equal to the appropriate version of Eqn. 3, depending on the magnitude of the adsorption energy, to determine the maximum nanoshell temperature, T. This calculation is summarized Table 1 for 50 nm diameter nanoshells with a 7 nm wall thickness, then for several types of particles in FIG. 16.

TABLE 1
A comparison of calculated temperatures attained by the gold nanoshells
due to plasmonic heating (for the same concentration of gold, 41.4 g/m³).

| | Laser | | | ε_{A}, | | |
|---|---|---|---|---|---|---|
| Duration of | energy | | A, gold | scattering | Absorbed | |
| laser | I₀ | Particle | sol | efficiency | energy | T, final |
| Irradiation | (μJ/ pulse) | morphology | absorbance | Q_{abs} | (J-gram⁻¹) | (° C.) |
| Initial, t = 0 | 680 | ~50 nm core, | 2.49 | ~0.8 | 284 | 1723 |
| | | 7 nm thick | | | | |
| after 10 min. | 680 | ~30 nm solid | 0.37 | ~0.98 | 193 | 1064 |
| | | particle | | | | |

The change in morphology after irradiation causes a drastic drop in the absorbance of the 800 nm wavelength light from a Ti: sapphire laser (pulse duration = 90 fs, repetition rate = 1 kHz).

The TEM images (FIG. 13) and UV/Vis spectra (FIG. 12) provide clear evidence that the gold nanoshells melt and/or anneal in response to NIR pulsed laser irradiation in agreement with the calculations in Table 1 and FIG. 16. The nanoshells are not small enough to exhibit appreciable melting point depression as predicted via the Kelvin equation, hence the nanoshells must be adsorbing sufficient energy to reach the bulk melting point of gold (1064° C.) From Table 1, and FIG. 16, the largest gold nanoshells are estimated to reach temperatures of ˜1700-1800° C., while the smaller nanoshells may reach the vaporization point of gold. The diameter of the nanoshell determines the absorption and scattering at 800 nm for a given shell thickness (FIG. 15). However, the mass of gold in the nanoshell increases with the square of the nanoshell diameter, which means that the energy needed to heat the nanoshell also increases with the square of the nanoshell radius. Hence, the optimal nanoshell size for maximum temperature rise is not the same as for maximum total energy absorption or duration of energy absorption (FIG. 14). FIG. 16 shows the surprising result that the largest particles with the best overlap with the NIR laser actually have the smallest temperature rise, even though they adsorb the greatest fraction of the incident light energy. The results in FIG. 16 for the maximum nanoshell temperature assume that dissipation of heat to the surrounding solution is slow compared to the time for plasmonic heating and that all the absorbed energy goes into increasing the nanoshell temperature.

To verify this assumption, the time scale for heat dissipation to the surrounding liquid was modeled as conduction from a spherical point source of total energy Q_{abs} (Q_{abs}=ρ_{g}VQ), to an infinite stagnant fluid of thermal diffusivity, α (for water, α=1.44×10⁻⁷ m²/sec):

$$\frac{\partial T}{\partial t} = \frac{α}{{r}^{2}} \frac{\partial }{\partial r} ( {r}^{2} \frac{\partial T}{\partial r} ) \qquad ( 4 a )$$

far from the nanoshell, the temperature is undisturbed (Tᵢ is the ambient temperature):

T(r→∞)=Tᵢ (4b)

and the approximate conservation of energy (ignoring the phase transitions of gold and water):

$${Q}_{abs} = {\int }_{0}^{∞} ρ {C}_{p} ( T ( r ) - {T}_{i} ) 4 π {r}^{2} d r \qquad ( 4 c )$$

in which Cₚ is the heat capacity (4180 J/kg−K) and ρ is the density (1000 kg/m³) of water. Losses by radiation were not considered, but would cause equilibration between the particle and the surrounding solution to happen faster. An approximate solution to Eqn. 4a that fits the boundary conditions (Eqn. 4b, c) is:

$$Δ T ( r , t ) = T ( r , t ) - {T}_{i} = \frac{{Q}_{abs}}{ρ {{C}_{p} ( 4 πα t )}^{3 / 2}} exp ⌊ - \frac{{r}^{2}}{4 α t} ⌋ \qquad ( 4 d )$$

If Eqn. 3a is used to determine ΔTₘₐₓ, the maximum change in temperature, ΔT (r,t) takes the form:

$$Δ T ( r , t ) = \frac{{ρ}_{g} {C}_{pg} V Δ {T}_{max}}{ρ {{C}_{p} ( 4 πα t )}^{3 / 2}} exp ⌊ - \frac{{r}^{2}}{4 α t} ⌋ \qquad ( 4 e )$$

The term in the denominator, (4παt)^{3/2} , corresponds to the volume of water affected by the heat pulse; Eqn. 4e is only valid for (4παt)^{3/2}>>V, the volume of the nanoshell. The heat pulse to the nanoshell dissipates as a Gaussian with a width of (2αt)^{1/2}, while the peak temperature (at r=0) decreases as V/(4παt)^{3/2}. τ, the time to approach equilibrium is that when ΔT/ΔTₘₐₓ˜0.001; for 50 nm diameter nanoshells with a shell thickness of 7 nm, V=4.1×10⁴ nm³, and τ−7 ns. While τ is short compared to the time between pulses, (1 ms), it is quite long compared to the 90 fs pulse length. Hence, the heat input to the nanoshell due to light adsorption is initially confined to the nanoshell, and the nanoshell relaxes to ambient temperature prior to the next light pulse. All of the heat input has gone into heating the surrounding water after about 10 ns.

FIG. 17 shows the measured temperature rise of 3 ml of suspension irradiated for 15 minutes with 680 μJ/pulse 800 nm NIR light at 1 kHz (0.68 W power). The initial heating rate is much larger than the rate after 15 minutes of irradiation, consistent with the increase in light transmission seen in FIG. 14 as the nanoshells melt, change shape, and the SPR resonance is blue shifted to the 500-600 nm resonance of solid spherical nanoparticles (FIG. 13). Most of the temperature rise occurs over the first minute or two, then the sample temperature reaches a steady state showing that the decreased energy input from adsorption is offset by losses from the suspension vial to the lab environment. If all 0.68 W of light energy was absorbed by the solution, the maximum heating rate of 3 ml of water would be ˜3.2° C./min; the initial heating rate of the largest nanoshells (0.41 mM Au) is ˜2.5° C./min and is decreases for the smaller nanoshells. The initial temperature increase of the solution, which is indicative of the total energy absorption of the nanoshells, increases with increasing nanoshell size (and overlap of the SPR peak with the excitation light). This order is reversed from the maximum temperature rise of the individual nanoshells (FIG. 16); the largest nanoshells are the most efficient energy absorbers, but have the smallest maximum temperature rise for the individual nanoshell (FIG. 16).

FIG. 14d shows that light absorption decreases significantly after 10 minutes irradiation (FIG. 14d), eventually leading to a steady state temperature at which losses from the sample vials equals the net rate of energy absorption. The steady state increase in temperature of the solution is proportional to the absorbance of the nanoparticles after the morphological and SPR resonance changes induced by the irradiation seen in FIG. 14d. The steady state temperature increases of the larger particles (3.2, 2.0 and 1.0° C.) are directly proportional to the gold concentration in the suspension (0.41 mM, 0.24 mM and 0.125 mM). Although the individual nanoparticles are heated to melting after the morphological changes (See FIG. 16), the steady state solution temperature increase is only a few degrees above ambient. As soon as the irradiation ends, the suspension quickly goes back to room temperature.

Heating the solution a second time (FIG. 17b) shows that the time to reach steady state more than doubles for the 40 nm nanoshell suspension (0.25 mM Au) than the first heating cycle. However, the steady state temperature increase was about 2° C. for both cycles. This is consistent with the TEM images in FIG. 13 that shows that the nanoshells have been degraded during the first irradiation and that the steady state temperature increase is due to off-resonance heating of the melted and annealed nanoshells (FIG. 12). The differences in steady state temperature in FIG. 17a primarily result from the differences in gold concentration in the solution, rather than the initial size of the gold nanoshells. By the time the suspension has reached a steady temperature, all of the nanoshells have been melted and annealed into stable, spherical solid nanoparticles with an SPR peak at 500-600 nm; only the total concentration of the melted nanoshells is different between the samples. Manipulating the particle morphologies to tune the SPR absorbance to the irradiation wavelength does not affect the steady state temperature of the suspensions when pulsed laser radiation is used. Similar on and off resonance heating of gold nanorods.

The emphasis of the synthesis presented here is on the facile and scalable nature of a new reaction pathway to gold nanoshells that (1) eliminates separation and centrifugation steps; (2) has minimal heating; and (3) has no toxic reactants, additives or solvents. The fact that gold nanoshells, or any other type of metallic or alloy nanoshell, can be made simply and rapidly via redox exchange chemistry (galvanic replacement) opens the door to a wide array of different materials chemistries and applications ranging from biomedical imaging to Raman spectroscopy to catalysis.

The previous work investigating the effect of pulsed laser irradiation of silica-gold nanoshells showed high temperatures consistent with the calculations and observations presented here for hollow gold nanoshells with an aqueous core. Those shells, however, were more of an aggregate structure and not a homogeneous shell. Hence, as has been observed in other studies, a complete gold shell is not necessary; aggregates of gold nanoparticles also have their absorption maximum shifted to the NIR. Hence, complete and defect-free nanoshells are not necessary for plasmonic heating applications.

The more unexpected result is that even nanoshells that have a SPR peak significantly removed from the 800 nm wavelength of the irradiating laser light can be heated to sufficiently high temperatures that they are quickly melted and annealed into more stable solid spherical nanoparticles. The energy absorbed by the nanoshells depends on the overlap between the SPR peak and the wavelength of the irradiation source, and hence on the diameter/shell thickness ratio. However, the maximum temperature of the nanoshells is limited by the mass of the nanoshells, which increases as the square of the diameter. Optimizing the nanoshell structure for maximum light absorption in the NIR does not necessarily provide the highest temperatures for the individual nanoshells during irradiation. It is not yet known if maximizing the nanoshell temperature or the energy absorption is more important for biomedical applications such as photothermal destruction of cancer cells. On continued irradiation with high power laser pulses, all of the nanoshells anneal to form solid, spherical nanoparticles of 10-40 nm diameter with a common SPR peak from 500 -600 nm. The steady state temperature rise is governed, as a result, only by the total gold concentration in the suspension.

Although a number of embodiments and features have been described above, it will be understood by those skilled in the art that modifications and variations of the described embodiments and features may be made without departing from the teachings of the disclosure or the scope of the subject matter as defined by the appended claims.

1. A method of generating monodisperse hollow metal nanostructures comprising:
contacting a template metallic nanostructure stabilized by biocompatible anions in an aqueous environment with a noble metal salt precursor having a greater standard reduction potential than the template metallic nanostructure.

2. A method of generating monodisperse hollow metal nanostructures comprising:
providing a template metal nanostructure;
modifying the size of the template metal nanostructure;
stabilizing the template metallic nanostructure with a biocompatible anion in an aqueous environment to provide a stabilized template metallic nanostructure;
adding a noble metal salt precursor to the stabilized template metallic nanostructure, wherein the noble metal salt precursor comprises a greater standard reduction potential than the template metallic nanostructure.

3. The method of claim 1 or 2, wherein the template metallic nanostructure comprises a metal of a lower reduction potential than the noble metal salt precursor.

4. The method of claim 1 or 2, wherein the biocompatible anion is citrate.

5. The method of claim 2, wherein the size of the template metallic nanostructure is modified to provide a desired Plasmon resonance.

6. The method of claim 1 or 2, wherein the template metallic nanostructure comprises a silver metal.

7. The method of claim 1 or 2, wherein the template metallic nanostructure comprises a geometry selected from the group consisting of a sphere, a cube, a tube, a triangle, a nanoring, and a bowl.

8. The method of claim 1 or 2, wherein the noble metal precursor salt is a selected from the group consisting of salts of gold, silver, platinum, ruthenium, rhodium, palladium and iridium.

9-10. (canceled)

11. The method of claim 1 or 2, wherein the template metallic nanostructure comprises a silver metal and the noble metal salt precursor comprises a gold noble salt.

12. The method of claim 1, wherein the noble metal salt precursor comprises HAuCl₄.

13. (canceled)

14. The method of claim 1, wherein the template metallic nanostructure is silver and the noble metal salt precursor is tetrachloroauric acid, the method comprising:
nucleating the template silver nanostructure with sodium citrate and silver nitrate,
modifying the size of the template silver nanostructure with the addition of silver nitrate and hydroxylamine hydrocholoride;
adding a desired amount of tetracholoroauric acid to the template silver nanostructure in an aqueous environment.

15. The method of claim 14, further comprising adding sodium borohydride during the nucleating process.

16. The method of claim 14, further comprising addition a reducing agent during modification of the size of the template silver nanostructure.

17. The method of claim 14, wherein the monodisperse hollow nanostructures are about 10-100 nm across.

18. (canceled)

19. A hollow nanostructure made by the method of claim 1.

20. The hollow nanostructure of claim 19, wherein the metallic nanostructure is a nanoshell.

21. The hollow nanostructure of claim 20, wherein the nanoshell is a hollow gold nanoshell or a metallic or metallic alloy material.

22. (canceled)

23. A composition comprising the nanostructure of claim 19 and a liposome.

24. The composition of claim 23, wherein the liposome encapsulates the nanostructure.

25. The composition of claim 23, wherein the liposome is tethered to the nanostructure.

26. The composition of claim 23, wherein the liposome comprises a diagnostic or therapeutic agent.

27. The composition of claim 23, wherein the liposome is selected from the group consisting of an MLV, a MVL, a ULV and a vesosome.

28-31. (canceled)

32. The composition of claim 23, wherein the nanostructure is selected from a shell, a particle, or a rod.

33. The composition of claim 23, further comprising a targeting moiety linked to the liposome.

34. The composition of claim 33, wherein the targeting moiety is an antibody, an antibody fragment, a receptor or a receptor ligand.

35. The composition of claim 26, wherein the therapeutic agent is a chemotherapeutic agent.

36. The composition of claim 23, further comprising a pharmaceutically acceptable carrier.

37. A formulation comprising:
a liposome;
a therapeutic or diagnostic agent encapsulated within the liposome;
a nanostructure;
wherein the nanostructure can absorb electromagnetic radiation and generate vibration or thermal energy from the electromagnetic radiation.

38. A method for delivery of an agent to a subject or tissue, comprising:
contacting the subject or tissue with a composition of claim 23;
contacting a desired location on the subject or tissue with an electromagnetic radiation comprising a wavelength that induces vibrational or thermal energy of the nanostructure for a sufficient time to cause a liposome in the composition to be disrupted.

39. The method of claim 38, wherein the liposome is selected from the group consisting of an MLV, a MVL, a ULV and a vesosome.

40-48. (canceled)

49. A method of treating a disease or disorder comprising contacting a subject in need of such treatment with a composition of claim 23 and contacting a site with electromagnetic radiation to cause the nanostructure to generate vibrational or thermal energy to disrupt the liposomes comprising the diagnostic or therapeutic agent.
